(19) United States
(12) Patent Application Publication
(10) Pub. No.: US 2016/0060700 A1
(43) Pub. Date: Mar. 3, 2016

(54) COPY NUMBER VARIATIONS AND AUTOIMMUNE DISEASES

(71) Applicant: Regents of the University of Minnesota, Minneapolis, MN (US)

(72) Inventor: Jianming Wu, Plymouth, MN (US)

(21) Appl. No.: 14/793,810

(22) Filed: Jul. 8, 2015

Related U.S. Application Data
(60) Provisional application No. 62/023,244, filed on Jul. 11, 2014.

(51) Int. Cl. C12Q 1/68

(52) U.S. Cl. CPC C12Q 1/6883; C12Q 2600/158; C12Q 2600/118

(57) ABSTRACT

This disclosure describes methods of determining the copy number of FCGR3A and/or FCGR3B in the genome of an individual, which is shown herein to be statistically significantly associated with an increased risk of the individual developing an autoimmune disease.

### CROSS REFERENCE TO RELATED APPLICATIONS

This application claims the benefit of priority under 35 U.S.C. 119(e) to U.S. Application No. 62/023,244, filed Jul. 11, 2014. This application is incorporated by reference in its entirety.

### FEDERALLY SPONSORED RESEARCH OR DEVELOPMENT

This invention was made with government support under R21-HL117652 awarded by the National Institutes of Health. The government has certain rights in the invention.

### BACKGROUND

Copy-number variations (CNVs) are structural alterations of the DNA of a genome that results in the cell having an abnormal variation in the number of copies of one or more sections of the DNA. CNVs correspond to relatively large regions of the genome that have been deleted or duplicated on certain chromosomes. Like other types of genetic variation such as single nucleotide polymorphisms (SNPs), certain CNVs have been associated with susceptibility or resistance to disease.

### SUMMARY

Methods of determining the copy number of FCGR3A and/or FCGR3B in the genome of an individual are described herein. The copy number of FCGR3A and FCGR3B has been shown herein to be statistically significantly associated with an increased risk of the individual developing an autoimmune disease.

In one aspect, a method of determining the copy number of FCGR3A and/or FCGR3B in an individual. Such a method typically includes providing a biological sample from the individual, and determining the copy number of FCGR3A and/or FCGR3B in the biological sample. Generally, a low copy number of FCGR3A indicates a statistically significantly increased risk factor for the individual developing an autoimmune disease such as, without limitation, SLE and/or RA. Generally, a low copy number of FCGR3B indicates a statistically significantly increased risk factor for the individual developing an autoimmune disease such as, without limitation, SLE.

In another aspect, a method of determining the risk of an individual for developing an autoimmune disease is provided. Such a method typically includes providing a biological sample from the individual; and determining the copy number of the FCGR3A gene and/or the FCGR3B gene in the biological sample. Generally, a low copy number of the FCGR3A gene and/or the FCGR3B gene is indicative of a statistically significantly increased risk for the individual to develop an autoimmune disease. Representative autoimmune diseases include, without limitation, SLE and/or RA. In some embodiments, low copy number refers to less than two copies.

In still another aspect, a method of determining the risk of an individual for developing systemic lupus erythematosus (SLE) and/or rheumatoid arthritis (RA) is provided. Such a method typically includes providing a biological sample from the individual, wherein the biological sample comprises DNA; and determining the copy number of the FCGR3A gene and/or the FCGR3B gene in the individual. Generally, a copy number of the FCGR3A gene of less than two is statistically significantly associated with an increased risk of the individual developing SLE and/or RA, and a copy number of the FCGR3B gene of less than two is statistically significantly associated with an increased risk of the individual developing SLE.

In some embodiments, the determining step uses PCR. In some embodiments, the determining step uses real-time PCR. In some embodiments, the primer sequences for determining the copy number of the FCGR3A gene are shown in SEQ ID NOs: 5 and 6. In some embodiments, the primer sequences for determining the copy number of the FCGR3B gene are shown in SEQ ID NOs: 8 and 9. In some embodiments, the probe sequence for determining the copy number of the FCGR3A gene is shown in SEQ ID NO:7. In some embodiments, the probe sequence for determining the copy number of the FCGR3B gene is shown in SEQ ID NO:10.

In some embodiments, the individual is Asian or of Asian descent. In some embodiments, the individual is Taiwanese or of Taiwanese decent.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 are graphs showing that FCGR3A and FCGR3B CN affect receptor expressions. CD16 expressions on NK cells and neutrophils were detected with FITC-conjugated anti-CD16 mAb as described herein and presented as MFI (mean fluorescent intensity). Panel A shows that FCGR3A CN affects CD16 expression on NK cells. Each solid dot (FCGR3A CN=1), square (FCGR3A CN=2), or triangle (CN=3) represents one human subject in the respective groups. NK cells carrying one copy (CN=1) of FCGR3A express significantly less CD16A than those carrying two copies (CN=2) of FCGR3A (P=0.0278). NK cells carrying three copies (CN=3) of FCGR3A also express significantly more CD16A than those with FCGR3A CN=1 (P=0.0401). Panel B shows that FCGR3B CN affects CD16 expression on neutrophils. Each solid dot (FCGR3B CN=1), square (FCGR3B CN=2), or triangle (FCGR3B CN=3) represents one human subject in the respective groups. Neutrophils from donors carrying FCGR3B CN=1 express significantly less CD16B than those from donors carrying FCGR3B CN=2 (P=0.005). In addition, neutrophils from donors carrying FCGR3B CN=3 express significantly more CD16B than those from donors carrying FCGR3B CN=2 (P=0.0048).

FIG. 2 are histograms of FCGR3A (Panel A) and FCGR3B (Panel B) CNV analysis in Copy Caller v2.0.

FIG. 3 is a schematic of the FCGR gene cluster.

### DETAILED DESCRIPTION

Methods are provided herein for determining the copy number of FCGR3A and/or FCGR3B in an individual. A statistically significant correlation is described herein between a low copy number of FCGR3A and/or FCGR3B and an increased risk for developing an autoimmune disease.

FCGR3A and FCGR3B are two of the five genes (in addition to FCGR2A, FCGR2B, and FCGR2C) in the FCGR gene cluster on chromosome 1 that encode IgG Fc receptors (FcγRs), which mediate a variety of immune functions that are critical in immune responses. Representative human FCGR3A and FCGR3B sequences can be found in, for example, GenBank Accession Nos. NG 009066.1 and NG 032926.1, respectively. Additional examples of FCGR3A and FCGR3B sequences can be found in the BAC clones shown in GenBank Accession No. AL590385, which includes both FCGR2A and FCGR3A, and GenBank Accession No. AL451067, which includes FCGR3A, FCGR2C, FCGR3B, and FCGR2B. An exemplary FCGR3A sequence is shown in SEQ ID NO:1 and encodes the polypeptide having the amino acid sequence shown in SEQ ID NO:2 (see Appendix B). An exemplary FCGR3B sequence is shown in SEQ ID NO:3 and encodes the polypeptide having the amino acid sequence shown in SEQ ID NO:4 (see Appendix B).

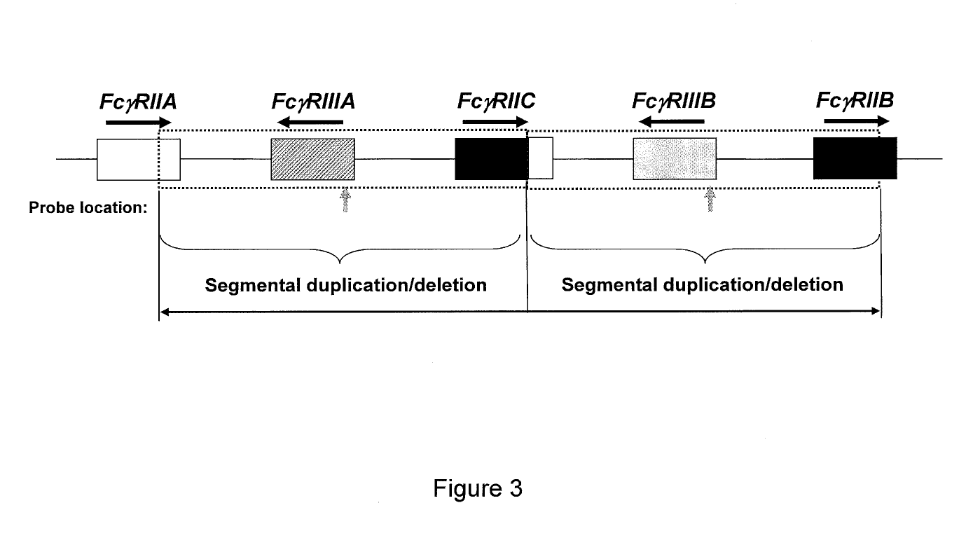

The FCGR gene cluster on chromosome 1q23 shows a complex pattern of copy number variations (CNVs). Of the five FCGR genes in the cluster (see FIG. 3), FCGR2C, FCGR3A, and FCGR3B exhibit CNVs while FCGR2A and FCGR2B do not exhibit CNVs. As used herein, copy number refers to the number of copies of FCGR3A or FCGR3B, while a low copy number generally refers to two copies or less (e.g., one copy).

Suitable biological samples are those that contain nucleic acid. As used herein, nucleic acids include DNA (e.g., cDNA or genomic DNA) and RNA. Typically, the biological sample contains DNA (e.g., genomic DNA) or RNA from the biological sample can be reverse transcribed into cDNA. A nucleic acid can be single stranded or double stranded, which usually depends upon its intended use. Copy number of FCGR3A and/or FCGR3B can be determined directly in the biological sample (i.e., without isolating the nucleic acid) or the nucleic acid can be isolated.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). An isolated nucleic acid molecule can be introduced into a vector (e.g., a cloning vector, or an expression vector) for convenience of manipulation or to generate a fusion molecule. In addition, an isolated nucleic acid molecule can include an engineered nucleic acid molecule such as a recombinant or a synthetic nucleic acid molecule.

Nucleic acids can be isolated using techniques routine in the art. For example, nucleic acids can be isolated using any method including, without limitation, recombinant nucleic acid technology and the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques that can be used to isolate a nucleic acid include, for example, restriction enzyme digestion and ligation. Isolated nucleic acids also can be chemically synthesized, either as a single nucleic acid molecule or as a series of oligonucleotides.

Similarly, a “purified” polypeptide is a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the polypeptides and naturally occurring molecules with which it is naturally associated. Since a polypeptide that is chemically synthesized is, by nature, separated from the components that naturally accompany it, a synthetic polypeptide is “purified.”

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector, or, for example, by chemical synthesis. The extent of purity of a polypeptide can be measured using any appropriate method, e.g., column chromatography, polyacrylamide gel electrophoresis, or HPLC analysis.

Autoimmune diseases are well known in the art and arise from an abnormal immune response of the body against substances and/or tissues normally present in the body. Autoimmune diseases include, without limitation, arthritis (e.g., rheumatoid arthritis (RA), chronic inflammatory arthritis, psoriatic arthritis, osteoarthritis), psoriasis, dermatitis, multiple sclerosis, inflammatory bowel disease (e.g., Crohn's disease, ulcerative colitis), lupus (e.g., systemic lupus erythematosis (SLE)), diabetes (e.g., Type I diabetes, Type II diabetes), sarcoidosis, myasthenia gravis, and amylotrophic lateral sclerosis (ALS). See, for example, Autoimmune Diseases [C20.111] at the US National Library of Medicine (at nlm.nih.gov/cgi/mesh/2011/MB_cgi?mode=&term=Autoimmune+Diseases on the World Wide Web).

Simply by way of example, systemic lupus erythematosus (SLE) and rheumatoid arthritis (RA) both are systemic inflammatory disorders. SLE primarily affects women during childbearing years. Disease manifestations are diverse and may range from non-specific symptoms such as fatigue and musculoskeletal complaints (e.g., arthralgia, myalgia) to life-threatening renal or cerebral disease.

As described herein, a low copy number of FCGR3A or a low copy number of FCGR3B is statistically significantly associated with an increased risk for developing an autoimmune disease. Specifically, a low copy number of FCGR3A is statistically significantly associated with an increased risk for developing SLE and/or RA, and a low copy number of FCGR3B is statistically significantly associated with an increased risk for developing SLE. Surprisingly, a high copy number of FCGR3A is statistically significantly associated with an increased risk for developing SLE, suggesting that an unbalanced FCGR3A (i.e., both deficiency and excess) play a role in the pathogenesis of SLE. As used herein, statistical significance refers to a p-value of less than 0.05, e.g., a p-value of less than 0.025 or a p-value of less than 0.01, using an appropriate measure of statistical significance, e.g., a one-tailed two sample t-test.

Copy number of FCGR3A and/or FCGR3B can be determined using any number of amplification techniques (see, e.g., PCR Primer: A Laboratory Manual, 1995, Dieffenbach & Dveksler, Eds., Cold Spring Harbor Laboratory Press, Cold Spring Harbor, N.Y.; and U.S. Pat. Nos. 4,683,195; 4,683,202; 4,800,159; and 4,965,188) with an appropriate pair of oligonucleotides (e.g., primers). A number of modifications to the original PCR have been developed and any suitable amplification method can be used to determine copy number.

For example, real-time PCR methods are provided that can be used to rapidly detect the copy number of FCGR3A and/or FCGR3B. Real-time PCR refers to methods in which PCR amplification and detection of the amplification product are combined in a single cuvette, thereby dramatically reducing the cycling time. Since detection occurs concurrently with amplification, the real-time PCR methods obviate the need for manipulation of the amplification product, and diminish the risk of cross-contamination between amplification reactions.

Briefly, amplification refers to cycling conditions that include multiple cycles of denaturing template nucleic acid, annealing oligonucleotide primers to the denatured template nucleic acid, and extension from the primers by a polymerase to produce an amplification product. Simply by way of example, the basic components of an amplification reaction mix can include about 10-25 nmole of each of the four deoxynucleoside triphosphates (e.g., dATP, dCTP, dTTP, and dGTP, or analogs thereof), 10-10 pmol of oligonucleotide primers, template nucleic acid, and a polymerase enzyme. The reaction components are generally suspended in a buffered aqueous solution having a pH of between about 7 and about 9, which can further include one or more co-factors required by the polymerase (e.g., Mg2+, K+). Additional components such as DMSO are optional. Template nucleic acid is typically denatured at a temperature of at least about 90° C., an extension from oligonucleotide primers is typically performed at a temperature of at least about 72° C.

The annealing temperature can be used to control the specificity of amplification. The temperature at which oligonucleotide primers anneal to template nucleic acid must be below the Tm of each of the primers, but high enough to avoid non-specific annealing of primers to the template nucleic acid. The Tm is the temperature at which half of the DNA duplexes have separated into single strands, and can be predicted for an oligonucleotide primer using the formula provided in section 11.46 of Sambook et al. (1989, Molecular Cloning: A Laboratory Manual, 2^{nd} Ed., Cold Spring Harbor Laboratory Press, Cold Spring Harbor, N.Y.).

In embodiments in which real-time PCR is used to determine copy number, each cycling step typically includes an amplification step and a hybridization step. The amplification step includes primer annealing and extension, and the hybridization step includes hybridization of one or more probes to the amplification product. Hybridization of the one or more probes can be detected using any number of means (e.g., fluorescence resonance energy transfer (FRET) using donor and corresponding acceptor fluorescent moieties; or double-stranded nucleic acid intercalating dyes such as, without limitation, SYBRGREEN or SYBRGOLD). A number of commercial systems are available for carrying out real-time PCR reactions. For example, LIGHTCYCLER (e.g., from Roche Molecular Biochemicals), TaqMan (e.g., from Applied Biosystems), or molecular beacon technologies can be used to detect copy number under real-time conditions.

It would be appreciated by those of skill in the art that appropriate positive and negative controls should be performed with every set of amplification reactions to avoid uncertainties related to contamination and/or non-specific annealing of oligonucleotide primers and extension therefrom.

Copy number of FCGR3A and/or FCGR3B also can be determined using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, N.Y.; Sections 7.37-7.57, 9.47-9.57, 11.7-11.8, and 11.45-11.57). The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed. Simply by way of example, high stringency conditions typically include a wash of the membranes in 0.2×SSC at 65° C.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane. Hybridization can be detected and, if desired, quantitated directly on a membrane or from an autoradiograph using, for example, a PhosphorImager or a Densitometer (Molecular Dynamics, Sunnyvale, Calif.).

Oligonucleotides (e.g., oligonucleotide primers or oligonucleotide probes) are usually from about 10 nucleotides to about 50 nucleotides in length (e.g., about 10 to 15, 10 to 25, 10 to 35, 10 to 40, 12 to 18, 12 to 20, 15 to 30, 15 to 45, 20 to 40, 25 to 35, 30 to 50, 35 to 50, or 40 to 50 nucleotides in length), but can be longer or shorter if appropriate amplification conditions are used. Oligonucleotide primers or probes can be designed using, for example, a computer program such as OLIGO (Molecular Biology Insights, Inc., Cascade, Colo.). Generally, individual oligonucleotide primers and probes are designed to have as little internal complementarity as possible (e.g., to avoid primer dimers). For purposes of amplification, a pair of primers generally is designed such that they act in combination to produce an amplification product; such design considerations include, without limitation, annealing to opposite strands of the template nucleic acid, having similar melting temperatures, and being an appropriate distance from one another such that the polymerase can effectively polymerize across the region and such that the amplification product can be readily detected using, for example, electrophoresis.

Representative oligonucleotides are shown, for example, in SEQ ID NOs: 5, 6, 7, 8, 9 and 10. Representative oligonucleotide primer sequences directed toward FCGR3A are shown in SEQ ID NOs: 5 and 6, while representative oligonucleotide primer sequences directed toward FCGR3B are shown in SEQ ID NOs: 8 and 9. For embodiments in which real-time PCR is used, a representative probe sequence directed toward FCGR3A is shown in SEQ ID NO:7, while a representative probe sequence directed toward FCGR3B is shown in SEQ ID NO:10.

It would be understood by a skilled artisan that an oligonucleotide primer or probe does not necessarily have to have a sequence that is identical (i.e., having 100% sequence identity) to the template or target nucleic acid. In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the computer program ClustalW and default parameters, which allows alignments of nucleic acid or polypeptide sequences to be carried out across their entire length (global alignment). Chenna et al., 2003, Nucleic Acids Res., 31(13):3497-500. ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the default parameters can be used (i.e., word size: 2; window size: 4; scoring method: percentage; number of top diagonals: 4; and gap penalty: 5); for an alignment of multiple nucleic acid sequences, the following parameters can be used: gap opening penalty: 10.0; gap extension penalty: 5.0; and weight transitions: yes. For fast pairwise alignment of polypeptide sequences, the following parameters can be used: word size: 1; window size: 5; scoring method: percentage; number of top diagonals: 5; and gap penalty: 3. For multiple alignment of polypeptide sequences, the following parameters can be used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; and residue-specific gap penalties: on. ClustalW can be run, for example, at the Baylor College of Medicine Search Launcher website or at the European Bioinformatics Institute website on the World Wide Web.

Detection (e.g., of an amplification product, of a hybridization complex) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

The methods described herein can be used to predict the risk of individuals who are Caucasian or of Caucasian descent, African American or of African American descent, Asian or of Asian descent, or Hispanic or of Hispanic descent, to develop an autoimmune disease. For example, the methods described herein can be used to predict the likelihood that an individual who is Asian or of Asian descent (e.g., Taiwanese or of Taiwanese decent) will develop an autoimmune disease such as, for example, SLE and/or RA. For example, the methods described herein can be used to predict the likelihood that an individual who is Caucasian or of Caucasian descent or African American or of African American descent will develop an autoimmune disease such as, for example, sarcoidosis. For example, the methods described herein can be used to predict the likelihood that an individual who is Caucasian or of Caucasian descent or African American or of African American descent will develop an autoimmune disease such as, for example, Alzheimer's disease.

Any of the methods described herein also can include an administering step. The administering step typically includes administering an effective amount of a therapeutic compound to the individual. The particular therapeutic compound as well as the effective amount can be determined from the results obtained in the determining step of the methods described herein (e.g., the copy number of FCGR3A and/or FCGR3B).

A variety of treatments are available, depending upon the particular autoimmune disease. Simply by way of example, treatment of SLE often includes immune modulating agents such as cyclosporine, hydroxychloroquine, corticosteroids, azathioprine, cyclophosphamide, mycophenolate mofetil, and intravenous immunoglobulin. Additionally, intravenous cyclophosphamide and prednisolone have been reported to be efficacious, and B cell depletion, biological agents, or hematopoietic stem cell transplants also can be used. In addition, there are three general classes of drugs commonly used in the treatment of rheumatoid arthritis: 1) non-steroidal anti-inflammatory agents (NSAIDs); 2) corticosteroids; and 3) disease modifying anti-rheumatic drugs (DMARDs). NSAIDs and corticosteroids have a short onset of action, while DMARDs can take several weeks or months to demonstrate a clinical effect. DMARDs include methotrexate, sulfasalazine, leflunomide, hydroxychloroquine, biological agents including T-cell costimulatory blockers, B-cell depletion antibodies, and TNFα inhibitors. Other immunomodulators are occasionally used, including azathioprine and cyclosporine.

Such compounds can be administered in an effective amount to an individual in need of such treatment (e.g., to an individual suffering from an autoimmune disease). Typically, an effective amount or a therapeutically effective amount or dose of a compound refers to the amount of compound that results in amelioration of symptoms or a prolongation of survival in a subject in the absence of toxicity.

Such compounds can be formulated with a pharmaceutically acceptable carrier for delivery (e.g., administration) to an individual. Pharmaceutically acceptable carriers are well known in the art. See, for example Remington: The Science and Practice of Pharmacy, University of the Sciences in Philadelphia, Ed., 21st Edition, 2005, Lippincott Williams & Wilkins; and The Pharmacological Basis of Therapeutics, Goodman and Gilman, Eds., 12th Ed., 2001, McGraw-Hill Co. The type of pharmaceutically acceptable carrier used in a particular formulation can depend on various factors, such as, for example, the physical and chemical properties of the compound, the route of administration, and the manufacturing procedure. Pharmaceutically acceptable carriers are available in the art, and include those listed in various pharmacopoeias. See, for example, the U.S. Pharmacopeia (USP), Japanese Pharmacopoeia (JP), European Pharmacopoeia (EP), and British pharmacopeia (BP); the U.S. Food and Drug Administration (FDA) Center for Drug Evaluation and Research (CDER) publications (e.g., Inactive Ingredient Guide (1996)); and Ash and Ash, Eds. (2002) Handbook of Pharmaceutical Additives, Synapse Information Resources, Inc., Endicott, N.Y.

Such compounds typically are formulated to be compatible with the intended route of administration. Suitable routes of administration include, for example, oral, rectal, topical, nasal, pulmonary, ocular, intestinal, and parenteral administration. Routes for parenteral administration include intravenous, intramuscular, and subcutaneous administration, as well as intraperitoneal, intra-arterial, intra-articular, intracardiac, intracisternal, intradermal, intralesional, intraocular, intrapleural, intrathecal, intrauterine, and intraventricular administration.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Study Subjects

Taiwanese healthy control donors (512 males and 908 females) have been recruited locally. The age of healthy control donors ranged from 18 to 64 years-old with a mean age of 40.2±11.6. Taiwanese SLE patients (72 males and 774 females) who fulfilled the 1982 and/or 1997 revised American College of Rheumatology criteria for SLE (Tan et al., 1982, Arth. Rheum., 25:1271-7) and RA patients (141 males and 807 females) who fulfilled the 1987 American Rheumatism Association criteria for RA (Arnett et al., 1988, Arth. Rheum., 31:315-24) were recruited at Chang Gung Memorial Hospital, Tao-Yuan, Taiwan. The stratifications of RA clinical characteristics were as previously described (Chen et al., 2011, J. Rheumatol., 38:264-70). The ethics committee of Chang Gung Memorial Hospital approved the human study and all donors provided written consent for the study.

### Example 2

### Nucleic Acid Isolation

Anti-coagulated peripheral blood was obtained from healthy control donors, SLE patients, and RA patients. All genomic DNA samples were isolated from anti-coagulated peripheral blood using the Puregene DNA isolation kit (Gentra Systems, Minneapolis, Minn.) in the same laboratory as previously described (Chen et al., 2006, Arth. Rheum., 54:3908-17).

### Example 3

### Determination of FCGR3 CNVs

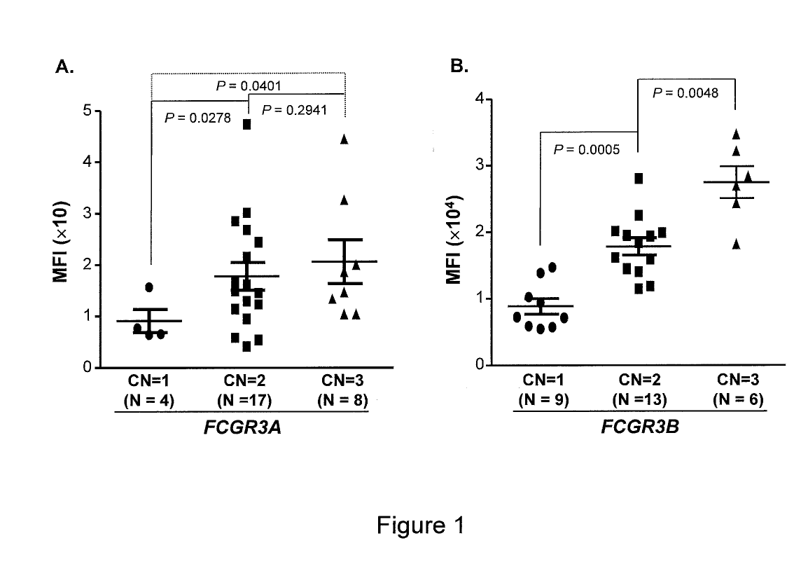

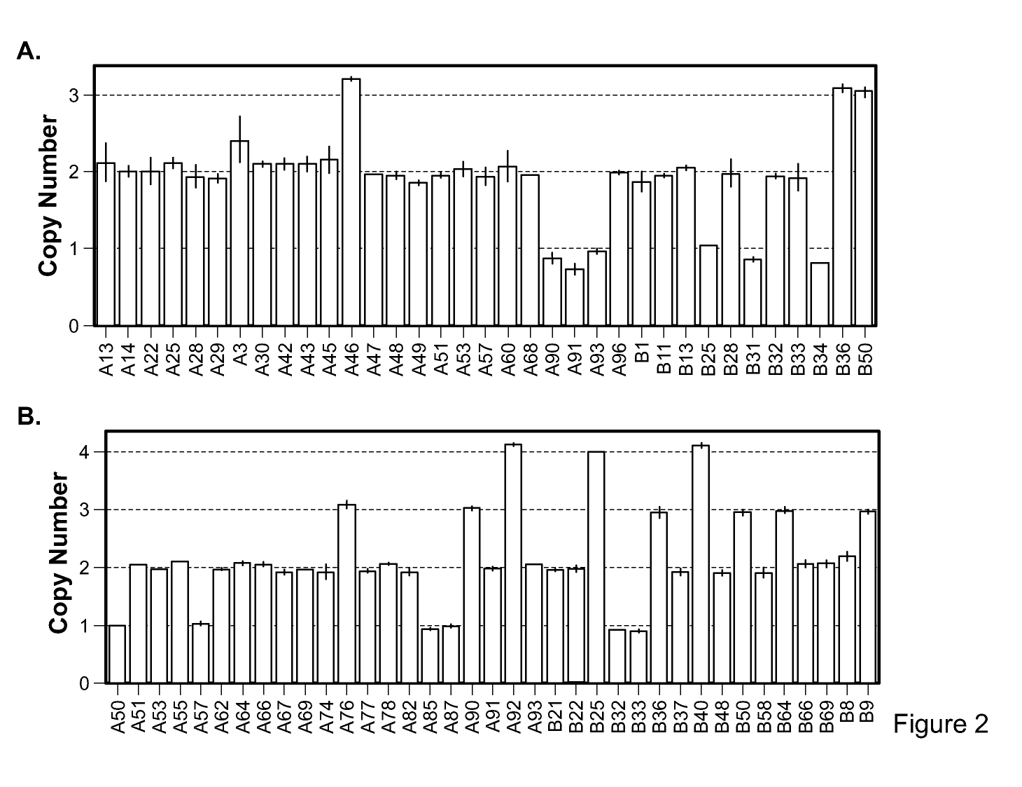

The CNVs of FCGR3A and FCGR3B were genotyped using custom TaqMan CNV real-time, quantitative PCR assays with FAM-MGB dual-labeled probes that were produced by Applied Biosystems (Foster City, Calif., USA) (Appendix A, Table 4). TaqMan Copy Number Reference Assay RNase P with VIC-TAMRA dual-labeled probe (Applied Biosystems, Cat #4403328) was used as the internal control of CN (copy number) reference. Duplex quantitative real-time PCR reactions were carried out on an Applied Biosystems ViiA 7 Real-Time PCR System (Life Technology) according to the manufacturer's instructions. All samples were tested in duplicate, and fluorescence signals were normalized to ROX. The quantitative PCR amplification curves were analyzed using ViiA 7 Software on a plate by plate basis, and the CN was assigned from the raw Cq values using COPYCALLER™ software (version 2.0; Applied Biosystems). This software employs a clustering algorithm and assigns the cluster with the most samples as CN=2. The COPYCALLER™ software also provides extensive diagnostics for the validity of the results, which were set to accept the CN assignment only when confidence was >95%, the standard deviation of the sample replicate ΔCq estimates was <0.20, and a reference gene Cq was <32. Over 85% of samples had more than 99% confidence level in the CN assignment. In addition, repeated copy number assay was carried out on all samples with CN<2, 10% healthy control samples with CN=2, and 20% all samples with CN>2 to confirm the CN calls. CN assignment with >95% confidence levels completely matched (100% reproducibility) for all samples in repeated CN assays. Overall, this methodology resulted in clear assignment of FCGR3 CN for 99% samples (the raw histograms of FCGR3A and FCGR3B CNV analyses are shown in FIG. 2). The accuracy of assays was further confirmed by determining CD16 expression on NK cells and neutrophils (FIG. 1).

### Example 4

### Evaluation of FcγRIII (CD16) Expression Levels

To determine expression of CD16 on NK cells and neutrophils, 100 μl fresh whole blood samples were stained with FITC-conjugated anti-human CD16 mAb (clone 16B) (eBioscience, San Diego, Calif.) and PE-conjugated anti-human CD56 (Beckman Coulter, Immunotech). Whole blood samples stained with FITC-conjugated mIgG1 and PE-conjugated anti-human CD56 in separate tubes were used as isotype controls. After incubation at room temperature for 30 min, blood samples were treated with 1×FACS Lysing Solution (BD Biosciences) to lyse red blood cells, followed by analysis on a Beckman Coulter FC 500 Flow cytometer. NK cells were identified within the lymphocyte population as CD56+ cells. Characteristic light-scatter properties were used to identify neutrophils in flow cytometry. Expression of FcγRIII (CD16) was analyzed with FlowJo software (Tree Star Inc.).

### Example 5

### Autoantibody Assay

Autoantibody titers were determined by ELISA. The autoantibody status was assessed at the time of SLE diagnosis. Antinuclear antibody (ANA) was considered as positive when serum titers were ≧1:80 in Hep-G2 cell assay. Anti-ENA (Ro/SSA, La/SSB, Sm, and RNP) and anti-cardiolipin antibodies were assessed by commercial ELISA according to vendor's instructions (Pharmacia Diagnostics).

### Example 6

### Statistical Analysis

The distributions of FCGR3 CN between patients (SLE or RA) and healthy controls were compared using χ2 test and Fisher's exact test. The P-value (P), odds ratio (OR), and 95% confidence interval (CI) were calculated based on the identified risk CN. To investigate the association of CN with clinical manifestations of SLE and RA, those SLE and RA patients with a phenotype were assigned a “1” and those without a phenotype were assigned a “0”. The clinical phenotypes of SLE patients were stratified according to SLE diagnosis criteria. The phenotypes of RA patients were stratified based on the presence or absence of RF, anti-CCP antibody, and radiographic erosions. The CN distributions were compared between “1” cases and “0” cases and between “1” cases and healthy controls using χ2 test and Fisher's exact test. In addition, logistic regression models adjusted for sex and age were used to investigate the relationship between each clinical manifestation and CN among patients. The logistic regression models adjusted for sex and age were also used to calculate ORs of high CN (>2) and low CN (<2) for disease susceptibility. To account for the multiple testing corrections, the False Discovery Rate (FDR)-adjusted P-values (Benjamini et al., 1995, J. Roy. Stat. Soc. B. Met., 57:289-300) were calculated with less conservative multipliers and control in a step-up fashion which was accomplished by using the SAS MULTTEST procedure. To evaluate the effect of FCGR3 CN on FcγRIII (CD16) expression levels, Mann-Whitney U test was used for the individuals with different copies of FCGR3. A P-value less than 0.05 was considered statistically significant in the study.

### Example 7

### Characteristics of SLE and RA Patients

The ages of 846 SLE patients (72 males and 774 females) ranged from 8 to 77 years-old with the mean age of 30.88±11.87 years. The mean age of 72 male SLE patients (8.51% of total SLE patients) was 31.46±12.52 years. The mean age of 774 female SLE patients (91.49% of total SLE patients) was 30.64±11.56 years. Descriptive clinical characteristics of 846 SLE patients are summarized in Appendix A, Table 1. The average age of 948 RA patients (141 males and 807 females) was 46.49±13.9 years. RA patients had the following characteristics: 76.4% patients were positive for CCP antibody (670/887), 78.81% patients were positive for RF (744/944), 50.9% patients were positive for ANA (424/833), and 71.04% patients manifested with destructive joints (601/846).

### Example 8

### Both FCGR3A and FCGR3B CNVs are Associated with SLE Susceptibility

The single-locus association was examined between the FCGR3 CNVs and the susceptibility to SLE. As shown in Appendix A, Table 2, the low FCGR3A CN (CN<2) was significantly associated with SLE disease susceptibility (CN<2 vs. CN=2, P=5.06±10⁻⁴, PFDR=0.001, OR 3.26, 95% CI: 1.68-6.35). Notably, the high FCGR3A CN (CN>2) was also a risk factor for SLE susceptibility (CN>2 vs. CN=2, P=0.003, PFDR=0.0061, OR 1.6, 95% CI: 1.17-2.18), suggesting that the abnormality of FCGR3A gene has a role in the development of SLE. Similarly, the low FCGR3B CN (CN<2) was significantly associated with SLE disease susceptibility (CN<2 vs. CN=2, P=0.0032, PFDR=0.0032, OR 1.59, 95% CI: 1.17-2.18). Moreover, the high FCGR3B CN (CN>2) tended to have a protective role against SLE disease development (Padjusted=0.0574, PFDR=0.0574, OR 0.77, 95% CI: 0.59-1.01; Appendix A, Table 2). The data suggest that the FcγRIIIB deficiency is also a risk factor for SLE in Taiwanese.

### Example 9

### RA Susceptibility is Associated with FCGR3A CNVs but not with FCGR3B CNVs

As shown in Appendix A, Table 3, the low FCGR3A CN (CN<2) was significant associated with RA disease susceptibility (CN<2 vs. CN=2, P=5.83±10-4, PFDR=0.0012, OR 2.82, 95% CI: 1.56-5.10). In contrast to the findings in SLE patients, the high FCGR3A CN (CN>2) had no effect on RA susceptibility (P=0.3335). In addition, neither low FCGR3B CN nor high FCGR3B CN was associated with RA susceptibility. The data indicate that FCGR3A deficiency is a susceptibility factor for RA while FCGR3B CNVs seem not to have a role in the development of RA in Taiwanese.

### Example 10

### Effects of FCGR3A and FCGR3B CNVs on SLE Phenotypes and Autoantibody Production

Human SLE patients exhibit heterogeneous manifestations and variations in the severity, nature, and spectrum of clinical involvement. The effects of FCGR3A and FCGR3B CNVs were subsequently examined on SLE clinical phenotypes. CN frequencies were compared between the SLE patients with each characteristic and the healthy controls (positive vs. healthy controls) and among SLE patients stratified by each clinical characteristic (positive vs. negative). Compared to the healthy controls, the low FCGR3A CN (CN<2) genotypes were significantly enriched in SLE patients positive for ulcer, arthritis, rash, discoid rash, photosensitivity, nephritis, leucopenia, thrombocytopenia, complement depression, anti-dsDNA, anti-RNP, anti-Sm, anti-SSA, anti-SSB, and anti-cardiolipin IgG (Appendix A, Table 5). However, only the low FCGR3A CN (CN<2) was marginally associated with nephritis among SLE patients stratified by clinical manifestations (nephritis positive SLE patients vs nephritis negative SLE patients, Padjusted=0.0457, OR 2.32, 95% CI: 1.02-5.28) (Appendix A, Table 6). The data suggests that FCGR3A deficiency may play a role in the development of lupus nephritis.

Compared to healthy controls, the low FCGR3B CN (CN<2) genotypes were significantly enriched in SLE patients with ulcer, rash, discoid rash, photosensitivity; ascites, nephritis, complement depression, and anti-dsDNA antibody production (Appendix A, Table 7). Nevertheless, only the phenotypes of oral ulcer and nephritis were associated with the low FCGR3B CN (CN<2) (PFDR<0.05) among SLE patients stratified by the clinical manifestations (Appendix A, Table 8).

### Example 11

### FCGR3 CNVs and RA Clinical Characteristics

It was also examined whether FCGR3A CNVs are associated with RA disease characteristics. Low FCGR3A CN (CN<2) was significantly increased in RA patients positive for RF and destructive RA as compared to the healthy controls (Appendix A, Table 9), but the enrichments were not significant when compared among the stratified RA patients (Appendix A, Table 10). On the other hand, the low FCGR3A CN (CN<2) was protective against anti-CCP antibody production (PFDR=0.008, OR 0.35, 95% CI: 0.17-0.72) among RA patients stratified by CCP positivity (Appendix A, Table 10). This data suggest that the functions of FcγRIIIA play a role in RA. However, FCGR3B was not associated with any RA clinical characteristics (Appendix A, Table 11 and 12).

Notably, low copy FCGR3A showed a negative association with anti-CCP antibody positivity, which highlighted a complicate role that FCGR3A CNVs play in autoimmune disease phenotypes and suggested that the genetic backgrounds may be different between RA patients positive for anti-CCP antibody and RA patients negative for anti-CCP antibody. Most importantly, FCGR3A deficiency is associated with two distinct autoimmune diseases (SLE and RA), suggesting that defective FcγRIIIA functions may be a common risk for various autoimmune diseases.

### Example 12

### FCGR3A CNVs Correlate with CD16A Expression on NK Cells

To evaluate whether the FCGR3A CNVs affect FcγRIIIA (CD16A) expression on NK cells, peripheral blood samples from individuals carrying one (n=4), two (n=17), and three (n=8) copies of FCGR3A were used in flow cytometry assays. As shown in FIG. 1A, NK cells carrying one copy (CN=1) of FCGR3A express significantly less CD16A than those carrying two copies (CN=2) of FCGR3A (P=0.028) and the NK cells carrying three copies (CN=3) of FCGR3A also express significantly more CD16A than those with FCGR3A CN=1 (P=0.040), suggesting that FCGR3A CNVs may affect NK cell functions.

As indicated herein, the data presented herein describes a correlation between low FCGR3A CN (CN=1) and low CD16A expression on NK cells (FIG. 1A), suggesting that FCGR3A CN has physiological implications in the NK cell functions. Therefore, modulating FcγRIIIA function likely will provide an important avenue for therapeutic treatment of lupus nephritis.

### Example 13

### FCGR3B CNVs Significantly Affect Neutrophil CD16B Expression

To assess the effect of FCGR3B CN on neutrophil FcγRIIIB (CD16B) expression, the CD16B expressions of individuals carrying one (n=9), two (n=13), or three (n=6) copies of FCGR3B was determined. As shown in FIG. 1B, neutrophils from individuals carrying one copy FCGR3B (CN=1) expressed significantly less CD16B than did the neutrophils from individuals having two copies of FCGR3B (CN=2) (P=0.0005). Meanwhile, the neutrophils from individuals carrying three copies of FCGR3B (CN=3) expressed significantly more CD16 than did the neutrophils from individuals having two copies of FCGR3B (P=0.0048). This data confirmed the previous observation that FCGR3B gene-dosages affect neutrophil CD16B expressions on neutrophils (Willcocks et al., 2008, J. Exp. Med., 205(7):1573-82).

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

TABLE 1
Clinical characteristics of the SLE study population (n = 846)

| Characteristics | Positive Cases/Total Cases (%) |
|---|---|
| Oral ulcer | 224/846 (26.48%) |
| Arthritis | 532/846 (62.88%) |
| Malar rash | 466/846 (55.08%) |
| Discoid rash | 161/846 (19.03%) |
| Photosensitivity | 189/846 (22.34%) |
| Pleural effusion | 162/846 (19.15%) |
| Pericardial effusion | 102/846 (12.06%) |
| Ascites | 44/846 (5.2%) |
| Nephritis | 471/846 (55.67%) |
| Neuropsychiatric manifestations | 135/846 (15.96%) |
| Leukopenia (WBC count <3500/uL) | 473/846 (55.91%) |
| Anemia (Hb <9 g/dL) | 257/846 (30.38%) |
| Thrombocytopenia (Plat count <10⁵/uL) | 220/846 (26%) |
| Anti-dsDNA | 628/828 (75.85%) |
| Complement depressed | 643/833 (77.19%) |
| Anti-RNP | 295/685 (43.07%) |
| Anti-Sm | 260/686 (37.9%) |
| Anti-SSA | 365/568 (64.26%) |
| Anti-SSB | 150/568 (26.41%) |
| Anti-cardiolipin IgG | 187/664 (28.16%) |
| Anti-cardiolipin IgM | 55/607 (9.06%) |

TABLE 2
Association of FCGR3A and FCGR3B CNVs with SLE susceptibility

| | | | | | | | Adjusted for sex & age | | | Adjusted for sex & age | | |
|---|---|---|---|---|---|---|---|---|---|---|---|---|
| | | | | | | | (CN > 2 vs CN = 2) | | | (CN < 2 vs CN = 2) | | |
| | | | | χ² Test | | Fisher's | | | OR | | | OR |
| Genes | CN < 2 | CN = 2 | CN > 2 | χ² | P | exact P | P | P_{FDR} | (95% CI) | P | P_{FDR} | (95% CI) |
| FCGR3A | | | | | | | | | | | | |
| SLE | 31 | 700 | 112 | 27.8166 | 9.11 × | 1.23 × | 0.0030 | 0.0061 | 1.6 | 5.06 × | 0.001 | 3.26 |
| | (3.68%) | (83.04%) | (13.29%) | | 10⁻⁷ | 10⁻⁶ | | | (1.17-2.18) | 10⁻⁴ | | (1.68-6.35) |
| Controls | 19 | 1280 | 121 | | | | | | | | | |
| | (1.34%) | (90.14%) | (8.52%) | | | | | | | | | |
| FCGR3B | | | | | | | | | | | | |
| SLE | 121 | 605 | 118 | 19.1486 | 6.95 × | 7.95 × | 0.0574 | 0.0574 | 0.77 | 0.0032 | 0.0032 | 1.59 |
| | (14.34%) | (71.68%) | (13.98%) | | 10⁻⁵ | 10⁻⁵ | | | (0.59-1.01) | | | (1.17-2.18) |
| Controls | 126 | 1026 | 253 | | | | | | | | | |
| | (8.97%) | (73.02%) | (18.01%) | | | | | | | | | |

The distributions of FCGR3 gene CNVs of SLE patients were compared to those of healthy controls using χ² and Fisher's exact tests.

TABLE 3
Association of FCGR3A and FCGR3B CNVs with RA susceptibility

| | | | | | | | Adjusted for sex & age | | | Adjusted for sex & age | | |
|---|---|---|---|---|---|---|---|---|---|---|---|---|
| | | | | | | | (CN > 2 vs CN = 2) | | | (CN < 2 vs CN = 2) | | |
| | | | | χ² Test | | Fisher's | | | OR | | | OR |
| Genes | CN < 2 | CN = 2 | CN > 2 | χ² | P | exact P | P | P_{FDR} | (95% CI) | P | P_{FDR} | (95% CI) |
| FCGR3A | | | | | | | | | | | | |
| RA | 36 | 836 | 73 | 15.5197 | 4.27 × | 5.00 × | 0.3335 | 0.432 | 0.86 | 5.83 × | 0.0012 | 2.82 |
| | (3.81%) | (88.47%) | (7.72%) | | 10⁻⁴ | 10⁻⁴ | | | (0.62-1.17) | 10⁻⁴ | | (1.56-5.1) |
| Controls | 19 | 1280 | 121 | | | | | | | | | |
| | (1.34%) | (90.14%) | (8.52%) | | | | | | | | | |
| FCGR3B | | | | | | | | | | | | |
| RA | 98 | 685 | 164 | 1.3208 | 0.5167 | 0.5160 | 0.4320 | 0.432 | 0.91 | 0.3584 | 0.3584 | 1.15 |
| | (10.35%) | (72.33%) | (17.32%) | | | | | | (0.72-1.15) | | | (0.85-1.55) |
| Controls | 126 | 1026 | 253 | | | | | | | | | |
| | (8.97%) | (73.02%) | (18.01%) | | | | | | | | | |

The distributions of FCGR3 gene CNVs of RA patients were compared to those of healthy controls using χ² and Fisher's exact tests.

TABLE 4
Primers and probes of TaqMan FCGR3A and FCGR 3B
CNV assays

| Gene | Gene-specific | TaqMan Probes |
|---|---|---|
| (SNP) | primers (5′ to 3′)* | (5′ to 3′)** |
| FCGR3A | 5′-TTC AAG AAA AGG | 5′-FAM-TCC ACT CCA |
| CNV | AAA TTG GTG-3′ | GTG TGG C-MGBNFQ-3′ |
| | (SEQ ID NO: 5); | (SEQ ID NO: 7) |
| | 5′-GAG GAG CAG CTG | |
| | CCA CAT G-3′ | |
| | (SEQ ID NO: 6) | |
| FCGR3B | 5′-TTC AAG AAA AGG | 5′-FAM-TCC ACT CCA |
| CNV | AAA CTG GCA-3′ | GTG TGG C-MGBNFQ-3′ |
| | (SEQ ID NO: 8) | (SEQ ID NO: 10) |
| | 5′-GAG GAG CAG CTG | |
| | CCA CAT G-3′ | |
| | (SEQ ID NO: 9) | |

*The sense primers of FCGR3A and FCGR3B CNV assays are specific for the target genes.
**The same FAM-6 labeled probe with minor groove binder (MGB) and non-fluorescent quencher (NFQ) on the 3′-end was used for FCGR3A and FCGR3B CNV assays in separate reactions.

TABLE 5
Association of FCGR3A CNVs with SLE clinical manifestations compared to the healthy controls

| | | | | | | | Adjusted for sex & age | | | Adjusted for sex & age | | |
|---|---|---|---|---|---|---|---|---|---|---|---|---|
| | | | | | | | (CN > 2 vs CN = 2) | | | (CN < 2 vs CN = 2) | | |
| | FCR3A CN | | | | | Fisher's | | | OR | | | OR |
| Subjects | CN < 2 | CN = 2 | CN > 2 | χ² | P | exact P | P | P_{FDR} | (95% CI) | P | P_{FDR} | (95% CI) |
| Healthy | 19 | 1280 | 121 | | | | | | | | | |
| controls | (1.34%) | (90.14%) | (8.52%) | | | | | | | | | |
| Ulcer⁺ | 10 | 193 | 21 | 11.23 | 0.0037 | 0.0081 | 0.5167 | 0.5167 | 1.19 | 0.0048 | 0.0048 | 3.6 |
| | (4.46%) | (86.16%) | (9.38%) | | | | | | (0.71-2) | | | (1.48-8.79) |
| Arthritis⁺ | 24 | 434 | 72 | 30.80 | 2.05 × | 5.73 × | 0.0125 | 0.0250 | 1.56 | 1.11 × | 0.0002 | 4.04 |
| | (4.53%) | (81.89%) | (13.58%) | | 10⁻⁷ | 10⁻⁷ | | | (1.1-2.22) | 10⁻⁴ | | (1.99-8.2) |
| Rash⁺ | 17 | 393 | 54 | 14.72 | 6.35 × | 9.81 × | 0.1316 | 0.1316 | 1.36 | 0.0019 | 0.0038 | 3.58 |
| | (3.66%) | (84.7%) | (11.64%) | | 10⁻⁴ | 10⁻⁴ | | | (0.91-2.02) | | | (1.6-8) |
| Discoid⁺ | 6 | 131 | 23 | 11.79 | 0.0027 | 0.0035 | 0.0363 | 0.0726 | 1.73 | 0.0224 | 0.0224 | 3.27 |
| | (3.75%) | (81.88%) | (14.38%) | | | | | | (1.04-2.9) | | | (1.18-9.02) |
| Photosens⁺ | 10 | 152 | 26 | 21.32 | 2.35 × | 1.27 × | 0.0211 | 0.0421 | 1.8 | 0.0014 | 0.0028 | 4.37 |
| | (5.32%) | (80.85%) | (13.83%) | | 10⁻⁵ | 10⁻⁴ | | | (1.09-2.96) | | | (1.77-10.79) |
| Pleural⁺ | 2 | 142 | 18 | 1.22 | 0.5428 | 0.5190 | 0.3245 | 0.3245 | 1.32 | 0.8257 | 0.8257 | 1.19 |
| | (1.23%) | (87.65%) | (11.11%) | | | | | | (0.76-2.29) | | | (0.26-5.44) |
| Pericardial⁺ | 3 | 83 | 16 | 7.94 | 0.0189 | 0.0155 | 0.0195 | 0.0391 | 2.03 | 0.0785 | 0.1570 | 3.22 |
| | (2.94%) | (81.37%) | (15.69%) | | | | | | (1.12-3.69) | | | (0.88-11.82) |
| Ascites⁺ | 2 | 38 | 3 | 3.32 | 0.1898 | 0.2009 | 0.7892 | 0.7892 | 0.85 | 0.0618 | 0.0618 | 4.51 |
| | (4.65%) | (88.37%) | (6.98%) | | | | | | (0.25-2.84) | | | (0.93-21.92) |
| Nephritis⁺ | 23 | 380 | 68 | 36.01 | 1.52 × | 7.08 × | 0.0010 | 0.002 | 1.84 | 4.06 × | <.0001 | 4.51 |
| | (4.88%) | (80.68%) | (14.44%) | | 10⁻⁸ | 10⁻⁸ | | | (1.28-2.64) | 10⁻⁸ | | (2.2-9.27) |
| CNS⁺ | 4 | 107 | 23 | 13.57 | 0.0011 | 0.0018 | 0.0022 | 0.0045 | 2.28 | 0.0980 | 0.1959 | 2.77 |
| | (2.99%) | (79.85%) | (17.16%) | | | | | | (1.34-3.87) | | | (0.83-9.29) |
| hema⁺ | 20 | 387 | 66 | 27.78 | 9.29 × | 2.45 × | 0.0012 | 0.0024 | 1.82 | 7.20 × | 0.0001 | 4.54 |
| | (4.23%) | (81.82%) | (13.95%) | | 10⁻⁷ | 10⁻⁶ | | | (1.27-2.61) | 10⁻⁵ | | (2.15-9.59) |
| Anemia⁺ | 7 | 216 | 34 | 8.82 | 0.0121 | 0.0117 | 0.0391 | 0.0781 | 1.61 | 0.0834 | 0.1668 | 2.4 |
| | (2.72%) | (84.05%) | (13.23%) | | | | | | (1.02-2.53) | | | (0.89-6.49) |
| Thromocyt⁺ | 11 | 182 | 26 | 17.50 | 1.58 × | 6.05 × | 0.0741 | 0.1482 | 1.56 | 4.71 × | 0.0009 | 4.66 |
| | (5.02%) | (83.11%) | (11.87%) | | 10⁻⁴ | 10⁻⁴ | | | (0.96-2.53) | 10⁻⁴ | | (1.97-11.06) |
| C3C4⁺ | 24 | 535 | 82 | 22.67 | 1.19 × | 2.06 × | 0.0155 | 0.0155 | 1.53 | 5.60 × | 0.0011 | 3.55 |
| | (3.74%) | (83.46%) | (12.79%) | | 10⁻⁵ | 10⁻⁵ | | | (1.08-2.15) | 10⁻⁴ | | (1.73-7.28) |
| Anti-dsDNA⁺ | 24 | 521 | 81 | 23.83 | 6.70 × | 1.16 × | 0.0103 | 0.0206 | 1.57 | 4.40 × | 0.0009 | 3.58 |
| | (3.83%) | (83.23%) | (12.94%) | | 10⁻⁶ | 10⁻⁵ | | | (1.11-2.21) | 10⁻⁴ | | (1.76-7.3) |
| Anti-RNP⁺ | 14 | 244 | 37 | 20.55 | 3.46 × | 1.23 × | 0.0696 | 0.1392 | 1.5 | 1.04 × | 0.0002 | 4.96 |
| | (4.75%) | (82.71%) | (12.54%) | | 10⁻⁵ | 10⁻⁴ | | | (0.97-2.33) | 10⁻⁴ | | (2.21-11.14) |
| Anti-Sm⁺ | 14 | 218 | 28 | 20.55 | 3.45 × | 1.91 × | 0.3005 | 0.6011 | 1.29 | 3.79 × | <.0001 | 5.58 |
| | (5.38%) | (83.85%) | (10.77%) | | 10⁻⁵ | 10⁻⁴ | | | (0.8-2.09) | 10⁻⁵ | | (2.46-12.63) |
| Anti-SSA⁺ | 17 | 296 | 51 | 27.55 | 1.04 × | 3.87 × | 0.0016 | 0.0033 | 1.86 | 1.65 × | 0.0003 | 4.27 |
| | (4.67%) | (81.32%) | (14.01%) | | 10⁻⁶ | 10⁻⁶ | | | (1.27-2.75) | 10⁻⁴ | | (2.01-9.1) |
| Anti-SSB⁺ | 7 | 116 | 27 | 24.51 | 4.76 × | 2.34 × | 2.14 × | 0.0004 | 2.55 | 0.0021 | 0.0041 | 4.76 |
| | (4.67%) | (77.33%) | (18%) | | 10⁻⁶ | 10⁻⁵ | 10⁻⁴ | | (1.55-4.18) | | | (1.76-12.84) |
| acaG⁺ | 6 | 151 | 29 | 14.05 | 8.90 × | 0.0012 | 0.0059 | 0.0119 | 1.96 | 0.0341 | 0.0683 | 3.06 |
| | (3.23%) | (81.18%) | (15.59%) | | 10⁻⁴ | | | | (1.21-3.16) | | | (1.09-8.61) |
| acaM⁺ | 0 | 46 | 9 | 4.68 | 0.0963 | 0.1421 | 0.0674 | 0.1348 | 2.05 | 0.9808 | 0.9808 | N/A |
| | (0%) | (83.64%) | (16.36%) | | | | | | (0.95-4.41) | | | |

CNV frequencies were compared between the SLE patients positive (denoted “+”) for each parameter and the healthy controls.
CNS: neuropsychiatric manifestations. hema: leucopenia. C3C4: complement depression. acaG: Anti-cardiolipin IgG. acaM: Anti-cardiolipin IgM.

TABLE 6
Association of FCGR3A CNVs with clinical manifestations among SLE patients

| | | | | | | | Adjusted for sex & age | | | Adjusted for sex & age | | |
|---|---|---|---|---|---|---|---|---|---|---|---|---|
| | | | | | | | (CN > 2 vs CN = 2) | | | (CN < 2 vs CN = 2) | | |
| | FCR3A CN | | | | | Fisher's | | | OR | | | OR |
| Subjects | CN < 2 | CN = 2 | CN > 2 | χ² | P | exact P | P | P_{FDR} | (95% CI) | P | P_{FDR} | (95% CI) |
| Ulcer = 1 | 10 | 193 | 21 | 4.38 | 0.1117 | 0.0981 | 0.0497 | 0.0994 | 0.6 | 0.5369 | 0.5369 | 1.28 |
| | (4.46%) | (86.16%) | (9.38%) | | | | | | (0.37-1) | | | (0.59-2.77) |
| Ulcer = 0 | 21 | 507 | 91 | | | | | | | | | |
| | (3.39%) | (81.91%) | (14.7%) | | | | | | | | | |
| Arthritis = 1 | 24 | 434 | 72 | 3.13 | 0.2086 | 0.2085 | 0.6544 | 0.7012 | 1.1 | 0.0781 | 0.1562 | 2.16 |
| | (4.53%) | (81.89%) | (13.58%) | | | | | | (0.73-1.67) | | | (0.92-5.1) |
| Arthritis = 0 | 7 | 266 | 40 | | | | | | | | | |
| | (2.24%) | (84.98%) | (12.78%) | | | | | | | | | |
| Rash = 1 | 17 | 393 | 54 | 2.45 | 0.2933 | 0.2862 | 0.0888 | 0.1046 | 0.7 | 0.7610 | 0.7610 | 0.89 |
| | (3.66%) | (84.7%) | (11.64%) | | | | | | (0.46-1.06) | | | (0.42-1.88) |
| Rash = 0 | 14 | 307 | 58 | | | | | | | | | |
| | (3.69%) | (81%) | (15.3%) | | | | | | | | | |
| Discoid = 1 | 6 | 131 | 23 | 0.21 | 0.8998 | 0.8910 | 0.6353 | 0.6353 | 1.13 | 0.9913 | 0.9913 | 1.01 |
| | (3.75%) | (81.88%) | (14.38%) | | | | | | (0.69-1.85) | | | (0.4-2.51) |
| Discoid = 0 | 25 | 569 | 89 | | | | | | | | | |
| | (3.66%) | (83.31%) | (13.03%) | | | | | | | | | |
| Photosens = 1 | 10 | 152 | 26 | 1.97 | 0.3741 | 0.3597 | 0.7358 | 0.7358 | 1.09 | 0.1751 | 0.2092 | 1.71 |
| | (5.32%) | (80.85%) | (13.83%) | | | | | | (0.68-1.74) | | | (0.79-3.73) |
| Photosens = 0 | 21 | 548 | 86 | | | | | | | | | |
| | (3.21%) | (83.66%) | (13.13%) | | | | | | | | | |
| Pleural = 1 | 2 | 142 | 18 | 4.48 | 0.1063 | 0.1066 | 0.3040 | 0.6080 | 0.75 | 0.0791 | 0.1582 | 0.27 |
| | (1.23%) | (87.65%) | (11.11%) | | | | | | (0.44-1.29) | | | (0.06-1.16) |
| Pleural = 0 | 29 | 558 | 94 | | | | | | | | | |
| | (4.26%) | (81.94%) | (13.8%) | | | | | | | | | |
| Pericardial = 1 | 3 | 83 | 16 | 0.71 | 0.7001 | 0.7587 | 0.4525 | 0.4525 | 1.25 | 0.6596 | 0.6596 | 0.76 |
| | (2.94%) | (81.37%) | (15.69%) | | | | | | (0.7-2.22) | | | (0.23-2.57) |
| Pericardial = 0 | 28 | 617 | 96 | | | | | | | | | |
| | (3.78%) | (83.27%) | (12.96%) | | | | | | | | | |
| Ascites = 1 | 2 | 38 | 3 | 1.63 | 0.4427 | 0.4507 | 0.2217 | 0.4434 | 0.48 | 0.8249 | 0.8249 | 1.18 |
| | (4.65%) | (88.37%) | (6.98%) | | | | | | (0.14-1.57) | | | (0.27-5.18) |
| Ascites = 0 | 29 | 662 | 109 | | | | | | | | | |
| | (3.63%) | (82.75%) | (13.63%) | | | | | | | | | |
| Nephritis = 1 | 23 | 380 | 68 | 6.00 | 0.0498 | 0.0495 | 0.2043 | 0.4086 | 1.3 | 0.0457 | 0.0457 | 2.32 |
| | (4.88%) | (80.68%) | (14.44%) | | | | | | (0.87-1.96) | | | (1.02-5.28) |
| Nephritis = 0 | 8 | 320 | 44 | | | | | | | | | |
| | (2.15%) | (86.02%) | (11.83%) | | | | | | | | | |
| CNS = 1 | 4 | 107 | 23 | 2.21 | 0.3318 | 0.3380 | 0.1700 | 0.1700 | 1.42 | 0.7119 | 0.7119 | 0.82 |
| | (2.99%) | (79.85%) | (17.16%) | | | | | | (0.86-2.35) | | | (0.28-2.39) |
| CNS = 0 | 27 | 593 | 89 | | | | | | | | | |
| | (3.81%) | (83.64%) | (12.55%) | | | | | | | | | |
| hema = 1 | 20 | 387 | 66 | 1.44 | 0.4858 | 0.5114 | 0.4752 | 0.5542 | 1.16 | 0.3232 | 0.3232 | 1.46 |
| | (4.23%) | (81.82%) | (13.95%) | | | | | | (0.77-1.74) | | | (0.69-3.1) |
| hema = 0* | 11 | 313 | 46 | | | | | | | | | |
| | (2.97%) | (84.59%) | (12.43%) | | | | | | | | | |
| Anemia = 1 | 7 | 216 | 34 | 0.96 | 0.6186 | 0.6690 | 0.8699 | 0.8911 | 0.96 | 0.3826 | 0.3826 | 0.68 |
| | (2.72%) | (84.05%) | (13.23%) | | | | | | (0.62-1.49) | | | (0.29-1.61) |
| Anemia = 0 | 24 | 484 | 78 | | | | | | | | | |
| | (4.1%) | (82.59%) | (13.31%) | | | | | | | | | |
| Thromocyt = 1 | 11 | 182 | 26 | 1.90 | 0.3864 | 0.3745 | 0.5383 | 0.5383 | 0.86 | 0.2877 | 0.4080 | 1.51 |
| | (5.02%) | (83.11%) | (11.87%) | | | | | | (0.54-1.38) | | | (0.71-3.22) |
| Thromocyt = 0 | 20 | 518 | 86 | | | | | | | | | |
| | (3.21%) | (83.01%) | (13.78%) | | | | | | | | | |
| C3C4 = 1 | 24 | 535 | 82 | 0.20 | 0.9055 | 0.9069 | 0.6620 | 0.6620 | 0.90 | 0.9172 | 0.9172 | 1.05 |
| | (3.74%) | (83.46%) | (12.79%) | | | | | | (0.56-1.44) | | | (0.44-2.5) |
| C3C4 = 0 | 6 | 155 | 28 | | | | | | | | | |
| | (3.17%) | (82.01%) | (14.81%) | | | | | | | | | |
| Anti- | 24 | 521 | 81 | 0.62 | 0.7340 | 0.7353 | 0.4675 | 0.4675 | 0.84 | 0.8552 | 0.8552 | 1.09 |
| dsDNA = 1 | (3.83%) | (83.23%) | (12.94%) | | | | | | (0.53-1.34) | | | (0.43-2.74) |
| Anti- | 7 | 164 | 28 | | | | | | | | | |
| dsDNA = 0 | (3.52%) | (82.41%) | (14.07%) | | | | | | | | | |
| Anti-RNP = 1 | 14 | 244 | 37 | 1.74 | 0.4189 | 0.4325 | 0.9885 | 0.9885 | 1 | 0.1882 | 0.3352 | 1.72 |
| | (4.75%) | (82.71%) | (12.54%) | | | | | | (0.63-1.59) | | | (0.77-3.87) |
| Anti-RNP = 0 | 11 | 328 | 48 | | | | | | | | | |
| | (2.84%) | (84.75%) | (12.4%) | | | | | | | | | |
| Anti-Sm = 1 | 14 | 218 | 28 | 4.21 | 0.1218 | 0.1217 | 0.3221 | 0.3221 | 0.78 | 0.0864 | 0.1104 | 2.04 |
| | (5.38%) | (83.85%) | (10.77%) | | | | | | (0.48-1.27) | | | (0.9-4.6) |
| Anti-Sm = 0 | 11 | 356 | 56 | | | | | | | | | |
| | (2.6%) | (84.16%) | (13.24%) | | | | | | | | | |
| Anti-SSA = 1 | 17 | 296 | 51 | 3.91 | 0.1418 | 0.1444 | 0.8412 | 0.8412 | 1.05 | 0.0602 | 0.1204 | 3.29 |
| | (4.67%) | (81.32%) | (14.01%) | | | | | | (0.64-1.73) | | | (0.95-11.4) |
| Anti-SSA = 0 | 3 | 171 | 28 | | | | | | | | | |
| | (1.49%) | (84.65%) | (13.86%) | | | | | | | | | |
| Anti-SSB = 1 | 7 | 116 | 27 | 3.29 | 0.1934 | 0.1922 | 0.0848 | 0.1696 | 1.57 | 0.5047 | 0.5047 | 1.37 |
| | (4.67%) | (77.33%) | (18%) | | | | | | (0.94-2.62) | | | (0.54-3.45) |
| Anti-SSB = 0 | 15 | 349 | 52 | | | | | | | | | |
| | (3.61%) | (83.89%) | (12.5%) | | | | | | | | | |
| acaG = 1 | 6 | 151 | 29 | 1.42 | 0.4927 | 0.5233 | 0.3163 | 0.6326 | 1.28 | 0.6584 | 0.9577 | 0.81 |
| | (3.23%) | (81.18%) | (15.59%) | | | | | | (0.79-2.08) | | | (0.32-2.06) |
| acaG = 0 | 20 | 396 | 59 | | | | | | | | | |
| | (4.21%) | (83.37%) | (12.42%) | | | | | | | | | |
| acaM = 1 | 0 | 46 | 9 | 2.97 | 0.2267 | 0.2367 | 0.6350 | 0.6616 | 1.2 | 0.9621 | 0.9621 | N/A |
| | (0%) | (83.64%) | (16.36%) | | | | | | (0.56-2.56) | | | |
| acaM = 0 | 26 | 450 | 73 | | | | | | | | | |
| | (4.74%) | (81.97%) | (13.3%) | | | | | | | | | |

CNV frequencies were compared between the SLE patients positive (denoted “1”) and negative (denoted “0”) for each clinical parameter.
CNS: neuropsychiatric manifestations. hema: leucopenia. C3C4: complement depression. acaG: Anti-cardiolipin gG. acaM: Anti-cardiolipin IgM.

TABLE 7
Association of FCGR3B CNVs with SLE clinical manifestations compared to the healthy controls

| | | | | | | | Adjusted for sex & age | | | Adjusted for sex & age | | |
|---|---|---|---|---|---|---|---|---|---|---|---|---|
| | | | | | | | (CN > 2 vs CN = 2) | | | (CN < 2 vs CN = 2) | | |
| | FCGR3B CN | | | | | Fisher's | | | OR | | | OR |
| Subjects | CN < 2 | CN = 2 | CN > 2 | χ² | P | exact P | P | P_{FDR} | (95% CI) | P | P_{FDR} | (95% CI) |
| Healthy | 126 | 1026 | 253 | | | | | | | | | |
| controls | (8.97%) | (73.02%) | (18.01%) | | | | | | | | | |
| Ulcer⁺ | 45 | 157 | 22 | 30.66 | 2.20 × | 9.13 × | 0.0138 | 0.0277 | 0.54 | 2.11 × | 0.0004 | 2.22 |
| | (20.09%) | (70.09%) | (9.82%) | | 10⁻⁷ | 10⁻⁷ | | | (0.33-0.88) | 10⁻⁴ | | (1.46-3.39) |
| Arthritis⁺ | 71 | 387 | 73 | 11.50 | 0.0032 | 0.0035 | 0.0498 | 0.0498 | 0.73 | 0.0587 | 0.0587 | 1.41 |
| | (13.37%) | (72.88%) | (13.75%) | | | | | | (0.53-1) | | | (0.99-2.02) |
| Rash⁺ | 70 | 339 | 56 | 19.87 | 4.85 × | 5.86 × | 0.0053 | 0.0105 | 0.6 | 0.0139 | 0.0139 | 1.6 |
| | (15.05%) | (72.9%) | (12.04%) | | 10⁻⁵ | 10⁻⁵ | | | (0.42-0.86) | | | (1.1-2.32) |
| Discoid⁺ | 30 | 106 | 25 | 15.08 | 5.32 × | 0.0015 | 0.7147 | 0.7147 | 0.91 | 0.0023 | 0.0046 | 2.13 |
| | (18.63%) | (65.84%) | (15.53%) | | 10⁻⁴ | | | | (0.57-1.48) | | | (1.31-3.46) |
| Photosens⁺ | 33 | 134 | 22 | 16.06 | 3.25 × | 6.43 × | 0.0528 | 0.0528 | 0.61 | 0.0123 | 0.0123 | 1.82 |
| | (17.46%) | (70.9%) | (11.64%) | | 10⁻⁴ | 10⁻⁴ | | | (0.37-1.01) | | | (1.14-2.9) |
| Pleural⁺ | 21 | 119 | 22 | 4.10 | 0.1289 | 0.1318 | 0.1276 | 0.2552 | 0.68 | 0.3313 | 0.6625 | 1.3 |
| | (12.96%) | (73.46%) | (13.58%) | | | | | | (0.42-1.12) | | | (0.77-2.2) |
| Pericardial⁺ | 12 | 71 | 19 | 0.99 | 0.6111 | 0.5692 | 0.9839 | 0.9839 | 1.01 | 0.3969 | 0.3969 | 1.33 |
| | (11.76%) | (69.61%) | (18.63%) | | | | | | (0.58-1.74) | | | (0.69-2.58) |
| Ascites⁺ | 11 | 26 | 6 | 13.46 | 0.0012 | 0.0044 | 0.7558 | 0.7892 | 0.86 | 0.0026 | 0.0053 | 3.21 |
| | (25.58%) | (60.47%) | (13.95%) | | | | | | (0.35-2.16) | | | (1.5-6.86) |
| Nephritis⁺ | 77 | 324 | 68 | 21.30 | 2.37 × | 4.70 × | 0.2233 | 0.2233 | 0.81 | 7.20 × | 0.0007 | 1.86 |
| | (16.42%) | (69.08%) | (14.5%) | | 10⁻⁵ | 10⁻⁵ | | | (0.59-1.13) | 10⁻⁴ | | (1.3-2.66) |
| CNS⁺ | 16 | 93 | 26 | 1.51 | 0.4706 | 0.4453 | 0.7244 | 0.7244 | 1.09 | 0.5597 | 0.5597 | 1.2 |
| | (11.85%) | (68.89%) | (19.26%) | | | | | | (0.67-1.76) | | | (0.65-2.19) |
| hema⁺ | 60 | 341 | 70 | 7.10 | 0.0287 | 0.0310 | 0.1125 | 0.1125 | 0.77 | 0.1070 | 0.107 | 1.36 |
| | (12.74%) | (72.4%) | (14.86%) | | | | | | (0.56-1.06) | | | (0.94-1.97) |
| Anemia⁺ | 29 | 190 | 37 | 2.90 | 0.2344 | 0.2274 | 0.1247 | 0.1247 | 0.73 | 0.6524 | 0.6524 | 1.12 |
| | (11.33%) | (74.22%) | (14.45%) | | | | | | (0.48-1.09) | | | (0.69-1.8) |
| Thromocyt⁺ | 27 | 156 | 37 | 2.47 | 0.2902 | 0.2957 | 0.6237 | 0.6237 | 0.9 | 0.2728 | 0.2728 | 1.31 |
| | (12.27%) | (70.91%) | (16.82%) | | | | | | (0.6-1.36) | | | (0.81-2.13) |
| C3C4⁺ | 91 | 471 | 79 | 11.94 | 0.0026 | 0.0027 | 0.0301 | 0.0301 | 0.71 | 0.0337 | 0.0337 | 1.45 |
| | (14.2%) | (73.48%) | (12.32%) | | | | | | (0.53-0.97) | | | (1.03-2.05) |
| Anti-dsDNA⁺ | 82 | 459 | 86 | 20.12 | 4.28 × | 4.51 × | 0.0032 | 0.0064 | 0.63 | 0.0166 | 0.0166 | 1.51 |
| | (13.08%) | (73.21%) | (13.72%) | | 10⁻⁵ | 10⁻⁵ | | | (0.46-0.86) | | | (1.08-2.12) |
| Anti-RNP⁺ | 36 | 215 | 43 | 4.33 | 0.1145 | 0.1160 | 0.1702 | 0.1702 | 0.76 | 0.1930 | 0.193 | 1.34 |
| | (12.24%) | (73.13%) | (14.63%) | | | | | | (0.52-1.12) | | | (0.86-2.1) |
| Anti-Sm⁺ | 28 | 185 | 45 | 0.93 | 0.6290 | 0.6123 | 0.7264 | 0.7264 | 0.93 | 0.3667 | 0.3667 | 1.25 |
| | (10.85%) | (71.71%) | (17.44%) | | | | | | (0.63-1.38) | | | (0.77-2.03) |
| Anti-SSA⁺ | 48 | 266 | 49 | 8.74 | 0.0126 | 0.0134 | 0.0424 | 0.0424 | 0.69 | 0.1581 | 0.1581 | 1.33 |
| | (13.22%) | (73.28%) | (13.5%) | | | | | | (0.48-0.99) | | | (0.9-1.98) |
| Anti-SSB⁺ | 23 | 108 | 18 | 8.60 | 0.0136 | 0.0169 | 0.0890 | 0.089 | 0.63 | 0.1037 | 0.1037 | 1.54 |
| | (15.44%) | (72.48%) | (12.08%) | | | | | | (0.37-1.07) | | | (0.92-2.6) |
| acaG⁺ | 27 | 134 | 26 | 6.77 | 0.0339 | 0.0399 | 0.2082 | 0.2082 | 0.74 | 0.1055 | 0.1055 | 1.51 |
| | (14.44%) | (71.66%) | (13.9%) | | | | | | (0.46-1.18) | | | (0.92-2.47) |
| acaM⁺ | 2 | 43 | 10 | 1.91 | 0.3850 | 0.4441 | 0.6805 | 0.6805 | 0.86 | 0.1495 | 0.2991 | 0.35 |
| | (3.64%) | (78.18%) | (18.18%) | | | | | | (0.42-1.77) | | | (0.08-1.47) |

CNV frequencies were compared between the SLE patients positive (denoted “+”) for each parameter and the healthy controls.
CNS: neuropsychiatric manifestations. hema: leucopenia. C3c4: complement depression. acaG: Anti-cardiolipin IgG. acaM: Anti-cardiolipin IgM.

TABLE 8
Association of FCGR3B CNVs with clinical manifestations among SLE patients

| | | | | | | | Adjusted for sex & age: | | | Adjusted for sex & age | | |
|---|---|---|---|---|---|---|---|---|---|---|---|---|
| | | | | | | | (CN > 2 vs CN = 2) | | | (CN < 2 vs CN = 2) | | |
| | FCGR3B CN | | | | | Fisher's | | | OR | | | OR |
| Subjects | CN < 2 | CN = 2 | CN > 2 | χ² | P | exact P | P | P_{FDR} | (95% CI) | P | P_{FDR} | (95% CI) |
| Ulcer = 1 | 45 | 157 | 22 | 10.92 | 0.0043 | 0.0046 | 0.1053 | 0.1053 | 0.66 | 0.0113 | 0.0225 | 1.7 |
| | (20.09%) | (70.09%) | (9.82%) | | | | | | (0.4-1.09) | | | (1.13-2.57) |
| Ulcer = 0 | 76 | 448 | 96 | | | | | | | | | |
| | (12.26%) | (72.26%) | (15.48%) | | | | | | | | | |
| Arthritis = 1 | 71 | 387 | 73 | 1.27 | 0.5289 | 0.5243 | 0.7012 | 0.7012 | 0.92 | 0.2835 | 0.2835 | 0.8 |
| | (13.37%) | (72.88%) | (13.75%) | | | | | | (0.61-1.39) | | | (0.54-1.2) |
| Arthritis = 0 | 50 | 218 | 45 | | | | | | | | | |
| | (15.97%) | (69.65%) | (14.38%) | | | | | | | | | |
| Rash = 1 | 70 | 339 | 56 | 3.37 | 0.1856 | 0.1872 | 0.1046 | 0.1046 | 0.71 | 0.6050 | 0.761 | 1.11 |
| | (15.05%) | (72.9%) | (12.04%) | | | | | | (0.47-1.07) | | | (0.74-1.67) |
| Rash = 0 | 51 | 266 | 62 | | | | | | | | | |
| | (13.46%) | (70.18%) | (16.36%) | | | | | | | | | |
| Discoid = 1 | 30 | 106 | 25 | 3.85 | 0.1458 | 0.1396 | 0.3654 | 0.6353 | 1.25 | 0.0650 | 0.1299 | 1.55 |
| | (18.63%) | (65.84%) | (15.53%) | | | | | | (0.77-2.05) | | | (0.97-2.47) |
| Discoid = 0 | 91 | 499 | 93 | | | | | | | | | |
| | (13.32%) | (73.06%) | (13.62%) | | | | | | | | | |
| Photosens = 1 | 33 | 134 | 22 | 2.63 | 0.2681 | 0.2722 | 0.4127 | 0.7358 | 0.81 | 0.2092 | 0.2092 | 1.33 |
| | (17.46%) | (70.9%) | (11.64%) | | | | | | (0.49-1.34) | | | (0.85-2.07) |
| Photosens = 0 | 88 | 471 | 96 | | | | | | | | | |
| | (13.44%) | (71.91%) | (14.66%) | | | | | | | | | |
| Pleural = 1 | 21 | 119 | 22 | 0.37 | 0.8291 | 0.8595 | 0.7964 | 0.7964 | 0.94 | 0.5234 | 0.5234 | 0.85 |
| | (12.96%) | (73.46%) | (13.58%) | | | | | | (0.56-1.55) | | | (0.51-1.41) |
| Pleural = 0 | 100 | 486 | 96 | | | | | | | | | |
| | (14.66%) | (71.26%) | (14.08%) | | | | | | | | | |
| Pericardial = 1 | 12 | 71 | 19 | 2.40 | 0.3017 | 0.3016 | 0.2084 | 0.4169 | 1.42 | 0.5149 | 0.6596 | 0.81 |
| | (11.76%) | (69.61%) | (18.63%) | | | | | | (0.82-2.47) | | | (0.42-1.54) |
| Pericardial = 0 | 109 | 534 | 99 | | | | | | | | | |
| | (14.69%) | (71.97%) | (13.34%) | | | | | | | | | |
| Ascites = 1 | 11 | 26 | 6 | 4.79 | 0.0911 | 0.0988 | 0.6678 | 0.6678 | 1.22 | 0.0304 | 0.0609 | 2.25 |
| | (25.58%) | (60.47%) | (13.95%) | | | | | | (0.49-3.04) | | | (1.08-4.7) |
| Ascites = 0 | 110 | 579 | 112 | | | | | | | | | |
| | (13.73%) | (72.28%) | (13.98%) | | | | | | | | | |
| Nephritis = 1 | 77 | 324 | 68 | 4.39 | 0.1115 | 0.1134 | 0.4115 | 0.4115 | 1.18 | 0.0385 | 0.0457 | 1.54 |
| | (16.42%) | (69.08%) | (14.5%) | | | | | | (0.79-1.77) | | | (1.02-2.31) |
| Nephritis = 0 | 44 | 281 | 50 | | | | | | | | | |
| | (11.73%) | (74.93%) | (13.33%) | | | | | | | | | |
| CNS = 1 | 16 | 93 | 26 | 4.07 | 0.1307 | 0.1444 | 0.0722 | 0.1445 | 1.57 | 0.6012 | 0.7119 | 0.86 |
| | (11.85%) | (68.89%) | (19.26%) | | | | | | (0.96-2.56) | | | (0.48-1.52) |
| CNS = 0 | 105 | 512 | 92 | | | | | | | | | |
| | (14.81%) | (72.21%) | (12.98%) | | | | | | | | | |
| hema = 1 | 60 | 341 | 70 | 2.57 | 0.2773 | 0.2853 | 0.5542 | 0.5542 | 1.13 | 0.1628 | 0.3232 | 0.76 |
| | (12.74%) | (72.4%) | (14.86%) | | | | | | (0.76-1.69) | | | (0.51-1.12) |
| hema = 0 | 61 | 264 | 48 | | | | | | | | | |
| | (16.35%) | (70.78%) | (12.87%) | | | | | | | | | |
| Anemia = 1 | 29 | 190 | 37 | 2.71 | 0.2582 | 0.2625 | 0.8911 | 0.8911 | 1.03 | 0.1270 | 0.254 | 0.7 |
| | (11.33%) | (74.22%) | (14.45%) | | | | | | (0.67-1.58) | | | (0.45-1.11) |
| Anemia = 0 | 92 | 415 | 81 | | | | | | | | | |
| | (15.65%) | (70.58%) | (13.78%) | | | | | | | | | |
| Thromocyt = 1 | 27 | 156 | 37 | 2.62 | 0.2695 | 0.2758 | 0.2272 | 0.4543 | 1.3 | 0.4080 | 0.408 | 0.82 |
| | (12.27%) | (70.91%) | (16.82%) | | | | | | (0.85-2.01) | | | (0.52-1.31) |
| Thromocyt = 0 | 94 | 449 | 81 | | | | | | | | | |
| | (15.06%) | (71.96%) | (12.98%) | | | | | | | | | |
| C3C4 = 1 | 91 | 471 | 79 | 2.85 | 0.2401 | 0.2369 | 0.5761 | 0.662 | 0.87 | 0.1136 | 0.2272 | 0.7 |
| | (14.2%) | (73.48%) | (12.32%) | | | | | | (0.55-1.4) | | | (0.45-1.09) |
| C3C4 = 0 | 28 | 127 | 35 | | | | | | | | | |
| | (14.74%) | (66.84%) | (18.42%) | | | | | | | | | |
| Anti-dsDNA = 1 | 82 | 459 | 86 | 4.90 | 0.0864 | 0.0935 | 0.0361 | 0.0721 | 0.62 | 0.6003 | 0.8552 | 0.88 |
| | (13.08%) | (73.21%) | (13.72%) | | | | | | (0.4-0.97) | | | (0.55-1.41) |
| Anti-dsDNA = 0 | 35 | 135 | 29 | | | | | | | | | |
| | (17.59%) | (67.84%) | (14.57%) | | | | | | | | | |
| Anti-RNP = 1 | 36 | 215 | 43 | 1.28 | 0.5265 | 0.5399 | 0.7568 | 0.9885 | 1.07 | 0.3352 | 0.3352 | 0.8 |
| | (12.24%) | (73.13%) | (14.63%) | | | | | | (0.69-1.67) | | | (0.51-1.26) |
| Anti-RNP = 0 | 59 | 278 | 52 | | | | | | | | | |
| | (15.17%) | (71.47%) | (13.37%) | | | | | | | | | |
| Anti-Sm = 1 | 28 | 185 | 45 | 6.66 | 0.0358 | 0.0368 | 0.1078 | 0.2156 | 1.44 | 0.1104 | 0.1104 | 0.68 |
| | (10.85%) | (71.71%) | (17.44%) | | | | | | (0.92-2.25) | | | (0.42-1.09) |
| Anti-Sm = 0 | 69 | 306 | 51 | | | | | | | | | |
| | (16.2%) | (71.83%) | (11.97%) | | | | | | | | | |
| Anti-SSA = 1 | 48 | 266 | 49 | 0.55 | 0.7614 | 0.7550 | 0.4566 | 0.8412 | 0.83 | 0.9948 | 0.9948 | 1 |
| | (13.22%) | (73.28%) | (13.5%) | | | | | | (0.51-1.35) | | | (0.59-1.68) |
| Anti-SSA = 0 | 26 | 145 | 32 | | | | | | | | | |
| | (12.81%) | (71.43%) | (15.76%) | | | | | | | | | |
| Anti-SSB = 1 | 23 | 108 | 18 | 1.43 | 0.4887 | 0.4897 | 0.4547 | 0.4547 | 0.8 | 0.3911 | 0.5047 | 1.27 |
| | (15.44%) | (72.48%) | (12.08%) | | | | | | (0.46-1.42) | | | (0.74-2.18) |
| Anti-SSB = 0 | 52 | 302 | 63 | | | | | | | | | |
| | (12.47%) | (72.42%) | (15.11%) | | | | | | | | | |
| acaG = 1 | 27 | 134 | 26 | 0.07 | 0.9657 | 0.9808 | 0.8142 | 0.8142 | 0.94 | 0.9577 | 0.9577 | 1.01 |
| | (14.44%) | (71.66%) | (13.9%) | | | | | | (0.58-1.54) | | | (0.62-1.65) |
| acaG = 0 | 68 | 338 | 70 | | | | | | | | | |
| | (14.29%) | (71.01%) | (14.71%) | | | | | | | | | |
| acaM = 1 | 2 | 43 | 10 | 6.60 | 0.0370 | 0.0192 | 0.6616 | 0.6616 | 1.18 | 0.0266 | 0.0531 | 0.2 |
| | (3.64%) | (78.18%) | (18.18%) | | | | | | (0.57-2.45) | | | (0.05-0.83) |
| acaM = 0 | 91 | 384 | 76 | | | | | | | | | |
| | (16.52%) | (69.69%) | (13.79%) | | | | | | | | | |

CNV frequencies were compared between the SLE patients positive (denoted “1”) and negative (denoted “0”) for each parameter.
CNS: neuropsychiatric manifestations. hema: leucopenia. C3C4: complement depression. acaG: Anti-cardiolipin IgG. acaM: Anti-cardiolipin IgM.

TABLE 9
Association of FCGR3A CNVs with RA clinical phenotypes compared to healthy controls

| | | | | | | | Adjusted for sex & age | | | Adjusted for sex & age | | |
|---|---|---|---|---|---|---|---|---|---|---|---|---|
| | | | | | | | (CN > 2 vs CN = 2) | | | (CN < 2 vs CN = 2) | | |
| | FCGR3A CN | | | | | Fisher's | | | OR | | | OR |
| Subjects | CN < 2 | CN = 2 | CN > 2 | χ² | P | exact P | P | P_{FDR} | (95% CI) | P | P_{FDR} | (95% CI) |
| Healthy | 19 | 1280 | 121 | | | | | | | | | |
| controls | (1.34%) | (90.14%) | (8.52%) | | | | | | | | | |
| ccp4⁺ | 18 | 606 | 44 | 6.8857 | 0.0320 | 0.0351 | 0.0682 | 0.1363 | 0.71 | 0.0717 | 0.1434 | 1.87 |
| | (2.69%) | (90.72%) | (6.59%) | | | | | | (0.49-1.03) | | | (0.95-3.69) |
| RF⁺ | 26 | 662 | 54 | 11.9622 | 0.0025 | 0.0033 | 0.1687 | 0.2327 | 0.78 | 0.0037 | 0.0074 | 2.54 |
| | (3.5%) | (89.22%) | (7.28%) | | | | | | (0.55-1.11) | | | (1.35-4.77) |
| ANA⁺ | 11 | 383 | 28 | 4.659 | 0.0973 | 0.1030 | 0.1318 | 0.2635 | 0.71 | 0.2725 | 0.3696 | 1.56 |
| | (2.61%) | (90.76%) | (6.64%) | | | | | | (0.46-1.11) | | | (0.71-3.43) |
| X-0des⁺ | 25 | 534 | 41 | 17.0525 | 1.98 × | 3.86 × | 0.1639 | 0.3277 | 0.76 | 3.83 × | 0.0008 | 3.16 |
| | (4.17%) | (89%) | (6.83%) | | 10⁻⁴ | 10⁻⁴ | | | (0.52-1.12) | 10⁻⁴ | | (1.67-5.95) |

CNV frequencies were compared between the RA patients positive for each phenotype (denoted “+”) and the healthy controls.
ccp4: CCP autoantibody detected by third generation CCP antibody detection kit. RF: rheumatoid factor. ANA: antinuclear antibody. X-0des: Destructive RA at the time of diagnosis.

TABLE 10
Association of FCGR3A CNVs with phenotypes among RA patients

| | | | | | | | Adjusted for sex & age | | | Adjusted for sex & age | | |
|---|---|---|---|---|---|---|---|---|---|---|---|---|
| | | | | | | | (CN > 2 vs CN = 2) | | | (CN < 2 vs CN = 2) | | |
| | FCGR3A CN | | | | | Fisher's | | | OR | | | OR |
| Subjects | CN < 2 | CN = 2 | CN > 2 | χ² | P | exact P | P | P_{FDR} | (95% CI) | P | P_{FDR} | (95% CI) |
| ccp4 = 1 | 18 | 606 | 44 | 9.56 | 0.0084 | 0.0120 | 0.5602 | 0.5602 | 0.84 | 3.98 × | 0.008 | 0.35 |
| | (2.69%) | (90.72%) | (6.59%) | | | | | | (0.46-1.52) | 10⁻³ | | (0.17-0.72) |
| ccp4 = 0 | 15 | 176 | 16 | | | | | | | | | |
| | (7.25%) | (85.02%) | (7.73%) | | | | | | | | | |
| RF = 1 | 26 | 662 | 54 | 1.78 | 0.4104 | 0.3776 | 0.4277 | 0.4277 | 0.8 | 0.3353 | 0.4876 | 0.69 |
| | (3.5%) | (89.22%) | (7.28%) | | | | | | (0.45-1.4) | | | (0.33-1.47) |
| RF = 0 | 10 | 171 | 18 | | | | | | | | | |
| | (5.03%) | (85.93%) | (9.05%) | | | | | | | | | |
| ANA = 1 | 11 | 383 | 28 | 3.29 | 0.1926 | 0.2039 | 0.2439 | 0.4878 | 0.73 | 0.1435 | 0.287 | 0.56 |
| | (2.61%) | (90.76%) | (6.64%) | | | | | | (0.44-1.23) | | | (0.26-1.21) |
| ANA = 0 | 18 | 355 | 35 | | | | | | | | | |
| | (4.41%) | (87.01%) | (8.58%) | | | | | | | | | |
| X-0des = 1 | 25 | 534 | 41 | 1.76 | 0.4144 | 0.4148 | 0.1917 | 0.3834 | 0.7 | 0.8388 | 0.8388 | 1.08 |
| | (4.17%) | (89%) | (6.83%) | | | | | | (0.41-1.2) | | | (0.5-2.36) |
| X-0des = 0 | 9 | 211 | 23 | | | | | | | | | |
| | (3.7%) | (86.83%) | (9.47%) | | | | | | | | | |

CNV frequencies were compared between the RA patients positive (denoted “1”) and negative (denoted “0”) for each phenotype.
ccp4: CCP autoantibody detected by third generation CCP antibody detection kit. RF: rheumatoid factor. ANA: antinuclear antibody. X-0des: Destructive RA at the time of diagnosis.

TABLE 11
Association of FCGR3B CNVs with RA clinical phenotypes compared to healthy controls

| | | | | | | | Logistic adjusted for sex & age | | | Logistic adjusted for sex & age | | |
|---|---|---|---|---|---|---|---|---|---|---|---|---|
| | | | | | | | (CN > 2 vs CN = 2) | | | (CN < 2 vs CN = 2) | | |
| | CNV Frequency | | | | | Fisher's | | | OR | | | OR |
| Subjects | CN < 2 | CN = 2 | CN > 2 | χ² | P | exact P | P | P_{FDR} | (95% CI) | P | P_{FDR} | (95% CI) |
| Healthy | 126 | 1026 | 253 | | | | | | | | | |
| controls | (8.97%) | (73.02%) | (18.01%) | | | | | | | | | |
| ccp4⁺ | 71 | 484 | 115 | 1.49 | 0.4751 | 0.4695 | 0.4066 | 0.4066 | 0.9 | 0.2901 | 0.2901 | 1.19 |
| | (10.6%) | (72.24%) | (17.16%) | | | | | | (0.69-1.16) | | | (0.86-1.65) |
| RF⁺ | 81 | 541 | 122 | 2.59 | 0.2741 | 0.2728 | 0.2327 | 0.2327 | 0.86 | 0.2060 | 0.206 | 1.23 |
| | (10.89%) | (72.72%) | (16.4%) | | | | | | (0.66-1.1) | | | (0.89-1.68) |
| ANA⁺ | 46 | 300 | 78 | 1.48 | 0.4761 | 0.4615 | 0.9923 | 0.9923 | 1 | 0.3696 | 0.3696 | 1.19 |
| | (10.85%) | (70.75%) | (18.4%) | | | | | | (0.74-1.34) | | | (0.81-1.74) |
| X-0des⁺ | 58 | 441 | 102 | 0.48 | 0.7882 | 0.7848 | 0.3701 | 0.3701 | 0.89 | 0.8012 | 0.8012 | 1.05 |
| | (9.65%) | (73.38%) | (16.97%) | | | | | | (0.68-1.16) | | | (0.74-1.48) |

CNV frequencies were compared between the RA patients positive for each phenotype (denoted “+”) and the healthy controls.
ccp4: CCP autoantibody detected by third generation CCP antibody detection kit. RF: rheumatoid factor. ANA: antinuclear antibody. X-0des: Destructive RA at the time of diagnosis.

TABLE 12
Association of FCGR3B CNVs with phenotypes among RA patients

| | | | | | | | Logistic adjusted for sex & age | | | Logistic adjusted for sex & age | | |
|---|---|---|---|---|---|---|---|---|---|---|---|---|
| | | | | | | | (CN > 2 vs CN = 2) | | | (CN < 2 vs CN = 2) | | |
| | CNV Frequency | | | | | Fisher's | | | OR | | | OR |
| Subjects | CN < 2 | CN = 2 | CN > 2 | χ² | P | exact P | P | P_{FDR} | (95% CI) | P | P_{FDR} | (95% CI) |
| ccp4 = 1 | 71 | 484 | 115 | 1.15 | 0.5628 | 0.5706 | 0.5238 | 0.5602 | 0.87 | 0.4416 | 0.4416 | 1.25 |
| | (10.6%) | (72.24%) | (17.16%) | | | | | | (0.58-1.32) | | | (0.71-2.19) |
| ccp4 = 0 | 17 | 150 | 39 | | | | | | | | | |
| | (8.25%) | (72.82%) | (18.93%) | | | | | | | | | |
| RF = 1 | 81 | 541 | 122 | 2.51 | 0.2848 | 0.2962 | 0.1293 | 0.2586 | 0.73 | 0.4876 | 0.4876 | 1.22 |
| | (10.89%) | (72.72%) | (16.4%) | | | | | | (0.49-1.1) | | | (0.7-2.13) |
| RF = 0 | 17 | 141 | 41 | | | | | | | | | |
| | (8.54%) | (70.85%) | (20.6%) | | | | | | | | | |
| ANA = 1 | 46 | 300 | 78 | 0.12 | 0.9407 | 0.9492 | 0.8024 | 0.8024 | 1.05 | 0.6330 | 0.633 | 1.12 |
| | (10.85%) | (70.75%) | (18.4%) | | | | | | (0.73-1.5) | | | (0.71-1.76) |
| ANA = 0 | 42 | 293 | 73 | | | | | | | | | |
| | (10.29%) | (71.81%) | (17.89%) | | | | | | | | | |
| X-0des = 1 | 58 | 441 | 102 | 0.78 | 0.6777 | 0.6543 | 0.4082 | 0.4082 | 0.85 | 0.7253 | 0.8388 | 0.91 |
| | (9.65%) | (73.38%) | (16.97%) | | | | | | (0.57-1.25) | | | (0.55-1.51) |
| X-0des = 0 | 25 | 172 | 47 | | | | | | | | | |
| | (10.25%) | (70.49%) | (19.26%) | | | | | | | | | |

CNV frequencies were compared between the RA patients positive (denoted “1”) and negative (denoted “0”) for each phenotype.
ccp4: CCP autoantibody detected by third generation CCP antibody detection kit. RF: rheumatoid factor. ANA: antinuclear antibody. X-0des: Destructive RA at the time of diagnosis.

APPENDIX B
FCGR3A:
ggagccccgg ctcctaggct gacagaccag cccagatcca gtggcccgga ggggcctgag ctaaatccgc
aggacctggg taacacgagg aaggtaaaga gttcctgtcc tcgcccctcc ccacccccac cttttctgtg
atcttttcag cctttcgctg gtgacttgtt cttccagggc ccatttctct accctacctg ggtttcttct
aacctggaaa tctaatgatc aaatcacact aaaaagtcag tagctcctgt ggattacata tcccaggagc
atatagattt tgaattttga attttgaaag aaattctgcg tggagataat attgaggcag agacactgct
agtggtctga agatttgaaa ggaccacttt ctgtgtgcag gcagggcctc agctggagat agatgggtct
gggcgaggca ggagagtgac aagttctgag gtgaaatgaa ggaagccctc agagaatgct cctcccacct
tgaatctcat ccccagggtc tcactgtccc attcttggtg ctgggtggat ccaaatccag gagatggggc
aagcatcctg ggatggctga gggcacactc tggcagattc tgtgtgtgtc ctcagatgct cagccacaga
cctttgaggg agtaaagggg gcagacccac ccaccttgcc tccaggctct ttccttcctg gtcctgttct
atggtggggc tcccttgcca gacttcagac tgagaagtca gatgaagttt caagaaaagg aaattggtgg
gtgacagaga tgggtggagg ggctggggaa aggctgttta cttcctcctg tctagtcggt ttggtccctt
tagggctccg gatatctttg gtgacttgtc cactccagtg tggcatcatg tggcagctgc tcctcccaac
tgctctgcta cttctaggta agtcagggtc tccctggttg agggagaagt ttgagatgcc ttgggttcag
cagagacccc ttttcaggct acgaatgaga ctcccacgaa gggatgggac ccctcaccac atctatagct
gtggattgag ctcctaggac aagccaagat ggggctagaa atgaggagaa tgctggttcc aattggggca
tactcatgag tgaggccagt cacttcaccc ctctgggtcc cagaatcact ctgtggaacc aaagagcttc
gactagatgg tccctagggt ctgtctcttt cagtttgaca ttccagggtt ctcctctatg attttcaatt
tctacccttt cttgtgggga tatgggttga ggctctttct gtagcttggt tcagggaaat tcaacctgta
cccttaattt gtgagtttgc acagggagca aggggtaagg gagcagtgtt gaaaataggg atttgtgttg
acagtggcgc aagaggcatg aacagtggag accagagagc aggtagcaag gtttccacca gaaacatcct
gattcttggg aaaattgggc tcctggggca gaggagggca ggggagtttt aaactcactc tatgttctaa
tcactctgat ctctgcccct actcaatatt tgatttatct tttttcttgc agtttcagct ggcatgcgga
ctggtgagtc agcttcatgg tcttggattg acccagtggg gcacatatgg ggacaaaggc cataagatat
tgggaaatgc ttgttgaatg ggaaaatgct gatgtggggt tagcagggat agttcctcca acacagcaga
acttggccct gtgcttctct ggccagcttt ccttaagata ctgaacaggc caaaaatggg gccaagatgc
tctaagactg agccaccaag catgggtttg caatgagctc attctggctt tgaggctccc tgggaatggc
agtgtagagc ctgctcctct ccctgtcctc accccacatt atcttggctc ctcagaagat ctcccaaagg
ctgtggtgtt cctggagcct caatggtaca gggtgctcga gaaggacagt gtgactctga agtgccaggg
agcctactcc cctgaggaca attccacaca gtggtttcac aatgagagcc tcatctcaag ccaggcctcg
agctacttca ttgacgctgc cacagtcgac gacagtggag agtacaggtg ccagacaaac ctctccaccc
tcagtgaccc ggtgcagcta gaagtccata tcggtgagtt gatgaagggg aagaggaaaa tcaccaataa
agggtgaaac aaagggtcct gaaatacttg gtaagagcca gagatgatat tcttagagat aaaagctaag
atgagatgat gtgtggtccc actgaatggt atcagagttg tagtcctagc tctaagtagg tcttgggcaa
aatgtcaaag cctgtcagac agtagatata ggactgctgc attgcacaat tccaagaatc cccatatgga
gtgcatacaa tgtgaatgtg tcatgtgaag gttaggccat ggcatagatg ctcaataata gttatttata
tatttatttt catttttttt aattttattt tttgagacag agtatcactc tgtcacccag gctggagtgc
aatgcggcaa tctcagctca ctgcaacttc tgcccccttg ggttgtagtg attctcctgc ctcagcctcc
cgagtagctg agattacagg cacccgccac cacgcccagc taatttttgt atttttagta gagacagggt
ttcaccatgt tggtcagtct ggtctcaaac tcctgacctc aggtgattca ccagccttgg cttcccaaag
tgctgggact acaggcgtga gccaccacac ctggccaata atatttattg aataaattaa tgaatttggt
gttaggacct caatctcctt ctcgctctca gacatgtaat gccctaagcc acctcccaaa gcaatcctag
tggcctagca tcatatcttt ctgtctcctc atcaatgcta tactcaaacc tataattaag cataaatttg
gtaatgtgat agctcttcca atagaggcag atacatgttc agcctgcaca ttaatcatga catgaaagtt
cttgtgtact attaacagaa tatagacgtc agacacaggt aggagaaata ttttgaaggc agaggtcttt
cctggtgtcc ctacaatctt accacatagg ctggtccctg cagtgtcgcc ctgcaaacct aactctactt
ccacggctgt tccattcata caatgtttat gggtggaaca agctttgggg gaagaagggc ataaggaggt
ggatctgcaa gagagctcca tggaattggg cctctgaaac tgatttttgt ggctctttgg cctctgacag
taccactcaa ctgacatggt cttcactctc cagagctaca agaagatatg tccatttcta gctaggtaag
agatgtccac ctacaaccaa ataaaatggg ggaattacca agagaaagca atagaaaaat caagtctaag
agttactagt ttgccttgaa cttggctcta gaaactggct ttagaagtct agccaatcaa ggctatatta
aactgtgacc atgagaatta gcttcaccag gtaaacttct gagcatcctt taatccttta ggacccattt
cacttatgtc ctcctctgag aagcattttt tacttctttt tttgtttgtt tgtttgtgtt tgtttttgtt
tttgtttttg agacagagtc tctctctgtc acccaagctg gagtgcagtg gcgcaatctt ggctcactgc
aacctccacc tcccgggttc aagcaattct cctgcctcag cctcccaagt agctgggact acaggtgcat
gccaccacgc ccggctaatt ttttgtattt ttagtagaga cagggtttcg cagcgttagc caggatggtc
ttgatctcct gacttcatga tctgcccacc tcggcctccc aaagtactag gattacagat gtgagccacc
gcgcccagcc tgcatttttt acttctttca ggcagaattt ctttattcca atctagtcag ccccgcagtc
ctttattctt agcctgttgt agcacttgtc atattgtatt gtgattattt ctgaatattt atgtttctat
gtctagactg tagattcttt gaggctgaga actatatgtc ccatcatctg ggtatctcca gtccacagtg
tgtcatacat agtgagtgct tgatgaaata tcacttgaag gaatatacat atggacattc actgggtcca
tgacaggata gattcgaaca agaatgttcc tccaaaggcc accagactat atactaacca tgactttatg
ctaataatga ttcatctctc tgctgaaaaa gtaagtggat agataggcac atggcttctt ttgataaatg
atatctctta ataggtaatg aagattactt tctgtttggc aaatctttgt ggtagagaat catgaccaac
acacgtccta ccaattttgt ttagcatcag gtagtagatt ttttaaatta tagtaattca agctgagaat
gtagatttaa aaaataaaat tattgtaaat tttgttttgt tcttattaca aaagtcattt ggggtcaatt
tcaaaaatat ataaaagtaa acaggagaaa tttaaaatgt ccttcagtcc cactccttca gagaaaaccc
ctgttaatat gtaagtgcat atccttcttt tttctgtgca taatactttt taaaatattt gaagtattat
gcttttttaa cttaaaattg tctcatgaat attttcttat gccattataa tacttaccta taacatcatt
attttttaat tattcaggcc ctttcccgac catgacctca tgttctctct ttgtgaagtc tgattacttg
gtgacatgat cgtgagaata agctctggcg atataagaat ttcctctctt gaaggccatg ctcagtaaat
tacttggtga catgatcgtg agaataagct ctggcgatac aagaatttcc tctcttgaag gccatgctca
gtaataaagt tggtctcacc gaggccctgt gacaccttag aaaccacgaa ttgccaggct gagcaatacc
agtcccgccc ttcccctccc tggtgtttac attgagttct ccttcacaat ttctgcagcc actccgtggc
caccgtcacc ttattcctga ctgccacaag agtctttcaa tattcctttg attgcctatt ccttctgaaa
tctacctttt cctctaatag ggcaattcat cattttcaaa tgcaattttt actctgatct agaacttact
gtgaatcctt gtcacctgcc acagcaaatc taagtctagc acttaaggat cctgcagata tgctcatcgt
tgcttctcac ttacctcatt gcttagtccc tctgctctaa ccctgtgtgt tgatcacatg tgtgtgtgtc
cctcttcccc attagacaaa ggtcttggta tgacttcagt tctcttgcag ggccccatca gctcttcccc
aaagggagct atgcagggtt gactcccaat ctggctttcc cttatgtctc aggatctggg tggtacgtgg
ccccttcaca aagctctgca ctgagagctg aggcctcccg ggcctggggt gtctgtgtct ttcaggctgg
ctgttgctcc aggcccctcg gtgggtgttc aaggaggaag accctattca cctgaggtgt cacagctgga
agaacactgc tctgcataag gtcacatatt tacagaatgg caaaggcagg aagtattttc atcataattc
tgacttctac attccaaaag ccacactcaa agacagcggc tcctacttct gcagggggct ttttgggagt
aaaaatgtgt cttcagagac tgtgaacatc accatcactc aaggtgagac atgtgccacc ctggaatgcc
cagggacgcc tgtgtgtgga acctgcaatc acactgggaa gttgagttgg gaggagattc ctgattctta
cacgcacttc ttcatatgtg gttccctcct ggtgatcacc aggaggtccc caaaagtccc tgattgcagg
gtaggtttgc agctctgttt cagtccattc ttttggggta gctaggaggt gtcattcact ctgcagcatg
atggcaggag cagaagccac atctcctccc caataaatac ctctgtcttt ccttacgcta atcacaccca
cggtgtcata tgttcctatc gtgctggcct ccttcttatc caagcctttt agccacgatc caaactggca
ggagcccctc atcccctcac agaaagagcc cagaacctgg gttctggccc tgcagctaat taaccatctg
accagaggtg agccacttag tctctctgaa ccccaatttc ttcttccgta acaaaaataa gctgacattt
attgggcacc tttcagtgtg ctagactctg tgctaaacaa ttctttacat gcacctggtt tgactatcac
agtagacctt cacaacatga gataggtaat attccatttt acagatgaag taaccgaggt gcaaaaataa
ataaataagt ttccctaagg tcacatcaaa gacttcaaag cctgtatatt taaccagtaa gtaaaagatt
tgaacaagca ctaatatcct atgatcccat taagtcatcc acaaaacatc tctaggttct gtagcaccag
cctccagaat cagagctcta gagtggtgtg cctggacttt ccagtttcac agaacttcta tctgtaacta
gcccaagaca taaattgtaa acaatttgca tgtagaaagg cagcaaaaca ccttttgaga ttttgacact
acaatgccat aatttgtaca aaaataattt catgacactt taaactgaaa gtaaatactc ccaagtggtt
agggaaagag agcaaataaa gcaaatgggg taacatgtaa acaatgagtg gatctgggta aaggatatac
gagattaaac tattctggtc attttttttt taagtttgga aatatatcaa aatcaagagt ttaaaaaatt
gaaatgcaaa atcaacaaat ttgtcccagt ttctagacca tagcattgtc tgacaatttc ttaactgtca
cacaaaaccc agcttacaac ctaacttgtt aacgctccct gtcacatctc tgtcaaacaa gcaggagcct
ttgctcagtg tttggtgagc tgtcctctgc tcagatagca ctaagatcag gaaccaatgg gaggaagcaa
tactttcccc cagacttccc caccattcct accacttgcc tgttggctgt tgtcaaagac tttctactgg
tgacctcact gtttgttcca aatatctgcc ttagtgactg tcattttttt tcatctctcc acttctccta
ataggtttgg cagtgtcaac catctcatca ttctttccac ctgggtacca agtctctttc tgcttggtga
tggtactcct ttttgcagtg gacacaggac tatatttctc tgtgaagaca aacattcgaa gctcaacaag
agactggaag gaccataaat ttaaatggag aaaggaccct caagacaaat gacccccatc ccatgggggt
aataagagca gtagcagcag catctctgaa catttctctg gatttgcaac cccatcatcc tcaggcctct
ctacaagcag caggaaacat agaactcaga gccagatccc ttatccaact ctcgactttt ccttggtctc
cagtggaagg gaaaagccca tgatcttcaa gcagggaagc cccagtgagt agctgcattc ctagaaattg
aagtttcaga gctacacaaa cactttttct gtcccaaccg ttccctcaca gcaaagcaac aatacaggct
agggatggta atcctttaaa catacaaaaa ttgctcgtgt tataaattac ccagtttaga ggggaaaaaa
aaacaattat tcctaaataa atggataagt agaattaatg gttgaggcag gaccatacag agtgtgggaa
ctgctgggga tctagggaat tcagtgggac caatgaaagc atggctgaga aatagcaggt agtccaggat
agtctaaggg aggtgttccc atctgagccc agagataagg gtgtcttcct agaacattag ccgtagtgga
attaacagga aatcatgagg gtgacgtaga attgagtctt ccaggggact ctatcagaac tggaccatct
ccaagtatat aacgatgagt cctcttaatg ctaggagtag aaaatggtcc taggaagggg actgaggatt
gcggtggggg gtggggtgga aaagaaagta cagaacaaac cctgtgtcac tgtcccaagt tgctaagtga
acagaactat ctcagcatca gaatgagaaa gcctgagaag aaagaaccaa ccacaagcac acaggaagga
aagcgcagga ggtgaaaatg ctttcttggc cagggtagta agaattagag gttaatgcag ggactgtaaa
accacctttt ctgcttcaat atctaattcc tgtgtagctt tgttcattgc atttattaaa caaatgttgt
ataaccaata ctaaatgtac tactgagctt cgctgagtta agttatgaaa ctttcaaatc cttcatcatg
tcagttccaa tgaggtgggg atggagaaga caattgttgc ttatgaaaga aagctttagc tgtctctgtt
ttgtaagctt taagcgcaac atttcttggt tccaataaag cattttacaa gatcttgcat gctactctta
gatagaagat gggaaaacca tggtaataaa atatgaatga taaaa (SEQ ID NO: 1)
MWQLLLPTALLLLVSAGMRTEDLPKAVVFLEPQWYRVLEKDSVTLKCQGAYSPEDNSTQWFHNESLISSQASSYFID
AATVDDSGEYRCQTNLSTLSDPVQLEVHIGWLLLQAPRWVFKEEDPIHLRCHSWKNTALHKVTYLQNGKGRKYFHHN
SDFYIPKATLKDSGSYFCRGLFGSKNVSSETVNITITQGLAVSTISSFFPPGYQVSFCLVMVLLFAVDTGLYFSVKT
NIRSSTRDWKDHKFKWRKDPQDK (SEQ ID NO: 2)
FCGR3B:
aggctgacag accagcccag atccagtggc ccggaggggc ctgagctaaa tccgcaggac ctgggtaaca
cgaggaaggt aaagagttcc tgtcctcacc cctccccacc cccacctttt ctgtgatctt ttcagccttt
cactggtgac ttgttcttcc agggcccatt tctctaccct acctgggttt cttctaacct ggaaatctaa
tgatcaaatc acactaaaaa gtcagctcct gtggattaca tatcccagga gcatatagat tttgaatttt
gaattttgaa agaaattctg cgtggagata atattgaggc agagacactg ctagtggtca aagatttgaa
aggacaactt tctgtgtgca ggcagggcct cggctggaga tagatgggtc tggacgaggc aggagagtga
gaagttctga ggtgaaatgc aggaagccct cagagaatgc tcctcccacc ttgaatctca tccccagggt
cttgctgtcc cattcttggt gctgggtgga tctaaatcca ggagatgggg gcaagcatcc tgggaaagct
gagggcacac tctggcagat tctgtgtgtg tcctcagatg ctcagccgca gacctttggg agagtaaagg
gggcacaccc acccaccttg cctccaggct ctttccttcc tattcctgtt ctatggtggg gctccattgc
gagacttcag attgagaaat cagatgaagt ttcaagaaaa ggaaactggc aggtgacaga gatgggtgga
gggactgggg aaaggctgtt tactccctcc tgtctagtcg gcttggtccc tttagggctc cggatatctt
tggtgacttg tccactccag tgtggcatca tgtggcagct gctcctccca actgctctgc tacttctagg
taagtcagga tctccctggt tgagggagaa gtttgagatg ccttgggttc atcagacacc ccttttcagg
ctacgaatga gactcccaca aagggatggg acccctcacc acatctatag ctgtggattg agctaccagg
acaagccaag atggggctag aaatgaggag aatgctggtt ccaattgggt catagtcatg agtgaggcca
gtcacttcac ccctctgggt cccagaatca ctatgtggaa ccaaagagct tcgactagat ggtccctagg
gtctgtctct ttcagtttga cattccaggg ttctcctcta tggtttttaa tttctaccct ttcttgtggg
gatatgggtt gaggctcttt ctgtagcttg gttcagggaa attcaacctg tacccttaat ttgtgagttt
gcacagggag caaggggtaa gggagcagtg ttgaaaatag ggatttgtgt tgacagtggc gcaagaggca
tgaacagtgg agaccagaga gcaggtagca aggtttccac cagaaacatc ctgattcttg ggaaaattgg
gctcctgggg cagaggaggg caggggagtt ttaaactcac tctatgttct aatcactctg atctctgccc
ctactcaata tttgatttac tcttttttct tgcagtttca gctggcatgc ggactggtga gtcagcttca
tggtcttgga ttgacccagt ggggcacata tggggacaat ggccataaga tattgggaaa tgcttgttga
atgggaaaat gctgatgtgg ggttagcagg gatagttcct ccaacacagc agaacttggc cctgtgcttc
tctggccagc tttccttaag atactgaaca ggccaaaaat ggggccaaga tgctctaaga ctgagccacc
aagcatgggt ttgcaatgag ctcattctgg ctttgaggct ccctgggaat ggcagtgtag agcctgctcc
tctccctgtc ctcaccccac attatcttgg ctcctcagaa gatctcccaa aggctgtggt gttcctggag
cctcaatggt acagcgtgct tgagaaggac agtgtgactc tgaagtgcca gggagcctac tcccctgagg
acaattccac acagtggttt cacaatgaga acctcatctc aagccaggcc tcgagctact tcattgacgc
tgccacagtc aacgacagtg gagagtacag gtgccagaca aacctctcca ccctcagtga cccggtgcag
ctagaagtcc atatcggtga gttgatgaag gggaagagga aaatcaccaa taaagggtga aacaaagggt
cctgaaatac ttggtaagag ccagagatga tattcttaga gataaaagct aagatgagat gatgtgtggt
cccactgaat ggtatcagag ttgtagtcct agctctaagt aggtcttggg caaaatgtca aagcctgtca
gacagtagat ataggctgct gcattgcaca attccaagaa tccccatatg gagtgcatac aatgtgaatg
tgtcatgtga aggttaggcc atggcataga tgctcaataa tagttattta tttatttatt ttcatttttt
ttaattttat tttttgagac agagtatcac tctctcaccc aggctggagt gcaatgcggc aatctgagct
cactgcaact tctgccccct tgggttgtag tgattctcca gcctcagcct cccgagtagc tgagattaca
ggcacccgcc accacgccca gctaattttt gtatttttag tagagacagg gtttcaccat gttggtcagt
ctggtctcaa actcctgacc tcaggtgatt caccggtctt ggctttccaa agtgctggga ctacaggcgt
gagccaccac acctggccaa taatttttat tgaataaatt aatgaatttg gtgttaggac ctcaatctcc
ttctcgctct cagacatgta atgccctaag ccacctccca aagcaatcct agtggcctag aatcatatct
ttctgtctcc tcatcaatgc tatactcaaa cctataatta agcataaatt tggtaatgtg atagctcttc
caatagaggc agatacatgt tcagcctgca cattaatcat gacatgaaag tccttgtgta ctattaacag
aatatagaca tcagacacag gtaggagaaa tattttgaag gcaggggtct ttcctggtgt ccctacaatc
ttaccacata gcctggtccc tgcagtgtcg ccctgcaaac ctaattctac ttccacggct gttccattca
tacaatgttt atgggtggaa caagctttgg gggaagaagg gcataaggag gtggatctgc aagagagctc
catggaattg ggcctctgaa actgattttt gtggctcttt ggcctctgac attaccactc aactgacatg
gtcttcactc tccagagcta caagaaggta tgtccatttc tagctaggta agagatgtcc acctacaacc
aaataaaatg ggggaattac caagagaaag caatagaaaa atcaagtcta ggagttacta gtttgccttg
aacttggctc tagaaactgg ctttagaagt ctagccaatc aaggctatat taaactgtga ccatgagaat
tagcttcacc aggtaaactt ctgagcatcc tttattcctt taggacccat ttcacttatg tcctcctctg
agaagcactt tttacttctt tgtttgtttg tttgtttttg tttttgtttt tgagacagag tttctctctg
tcacccaagc tggagtgcag tggcgcaatc ttggttcact gcaacctcca cctccctggt tccagcgatt
ctcctgcttc agcctcccaa ctagctggga ctacaggtgc atgccaccac gcccggctaa ttttttgtat
ttttagtaga gacagggttt cacggcatta gccaggatgg tcttgatctc ctgacctcgt gatctgccta
cttcagcctc ccaaagtact aggattacag atgtgagcca ccgcgcccag cctgcatttt ttacttcttt
caggcagaat ttctttattc caatctagtc ggccccgcag tcctttattc ttagcctgtt gtagcactcg
tcatattgta ttgtgattat ttctgaatat ttatgtttct atgtctaggc tgtagattct ttgaggctga
gaactatatg tcccatcatc tgggtatctc cagtccacag tgtgtcatac atagtgagtg cttgatgaaa
tatcacttga aggaatatac acatgggcat tcactgagtc catgacagga cagattcgaa caagaatgtt
cctccaaagg ccaccagact atatactaac catgacttta tgctaataat gattcatctc tttgctgaaa
aagtaagtgg atagataggc acatggcttc ttttgataaa tgatatctct taataggtaa tgaagattac
tttctgtttg gcaaatcttt gtgttagaga atcatgacca acacacgtcc taccaatttt gtttagcatc
aggtagtaga tttttaaaat tatagtaatt caacctgaga atgtagattt aaaaaataaa attacagtaa
atttagtttt gctcttatta cgtttcgggt caatttcaaa aatatataaa agtaaacagg agaaatttaa
aatgtccttt ggtcccactc cttcagagaa aacccctgtt aatatgtaag tgcatatcct tcttttttct
gtgcataata ctttttaaaa tatttgaagt attatgcttt tttaacttaa cattgtctca tgaatatttt
cttatgccat tataatactt acctataaca tcattatttt tttaattatt caggccattt cccgaccatg
acctcatgtt ctctctttgt gaagtctgat tacttggtga catgattgtg agaataagct ctggcgatat
aagaatttcc tctcttgaag gccatgctca gtaataaagt tggtctcacc gaggccctgt gacaccttag
aaaccacgaa ttgccactct gagcactacc agtcccgccc ttcgccaccc tgatgtttac attgagttag
ttctccttca caatttctgc agccactctg tggcgaccgt cagcttattc ctgaaggcca caagagtctt
tcaatattcc tttgattgcc tattccttct gaaatctacc ttttcctcta atagagcaac tcatcatttt
caaatgcaat ttttactctg atctagaact tactgtgaat ccttgtcacc tgccacagca aatctaagtc
tagcacttaa ggatcctgca gatatgctca tcgttgcttc tcacttacct cattgcttag tccctctgct
ctaaccctgt gtgttgatca catgtgtgtg tgtccctctt ccccattaga caaaggtctt ggtatgactt
cagttctctt gcagggcccc atcagctctt ccccaaaggg agctatgcag ggttgactcc caatctggct
ttcccttatg cctcaggatc tgggtggtac gtggcccctt cacaaagctc tgcactgaga gctgaggcct
cccgggcctg gggtgtctct gtgtctttca ggctggctgt tgctccaggc ccctcggtgg gtgttcaagg
aggaagaccc tattcacctg aggtgtcaca gctggaagaa cactgctctg cataaggtca catatttaca
gaatggcaaa gacaggaagt attttcatca taattctgac ttccacattc caaaagccac actcaaagat
agcggctcct acttctgcag ggggcttgtt gggagtaaaa atgtgtcttc agagactgtg aacatcacca
tcactcaagg tgagacatgt gccaccctgg aatgcccagg gacgcctgtg tgtggaacct gcactcacac
tgggaagttg agttgggagg agattcctga ttcttacacg cacttcttca tatgtggttc cctcctggtg
atcaccagga ggtccccaaa agtccctgat tgcagggtag gtttgcagct ctgtttcagt ccattctttt
ggggtagcta ggaggtgtca ttcactctgc agcatgatgg caggagcaga agccacatct cctccccaat
aaatacctct gtctttcctt accctaatca cacccacggt gtcatatgtt cctatcgtgc tggcctcctt
cttatccaag ccttttagcc acgatccaaa ctggcaggag cccctcatcc cctcacagaa agagcccaga
acctgggttc tggccctgca gctaattaac catctgacca gaggtgagcc acttagtctc tctgaacccc
aatttcttct tccgtaacaa aaataagctg acatttattg ggcacctttc agtgtgctag actctgtgct
aaacaattct ttacatgcac ctggtttgac taccacagta gaccttcaca acatgagata ggtaatattc
cattttacag atgaagtaac cgaggtgcaa aaataaataa ataagttgcc cctaaggtca catcaaagac
ttcaaagcct gtatatttaa ccagtaagta aaagatttga agaagcacta atatcctatg atcccattaa
gtcatccaca aaacatctct aggttccata gcaccagcct ccagaatcag agctctagag tggtgtgcct
ggactttcca gtttcacaga acttctatct gtaactagcc caagacataa attgtaaaca atttgcatgt
agaaaggcag caaaacacct tttgagattt tgacaccaca atgccataat ttgtacaaaa ataatttcat
gacactttaa actgaaagta aatactccca agtggttagg gaaagagagc aaataaagca aatggggtaa
catgtaaaca atgagtggat ctgggtaaag gatatacgag atttctttat actattctgg tcattttttt
taagtttgga aatatatcaa aatcaagagt ttaaaaaatt gaaatgcaaa atcaacaaat ttatcccagt
ttctagacca tagcattgtc tgacaatttc ttaactgtca cacaaaaccc agcttacaac ctaacttgtt
aacgctccct gtcacatctc tgtcaaacaa gcaggagcct ttgctaagtg tttggtgagc tgtcctctgc
tcagatagca ctaagatcag gaaccaatgg gaggaagcaa tactttcccc cagacctccc caccattcct
accacttgcc tgttggcact tgtcaaagac tctctactgg tgacctcact gtttgttcca aatatctgcc
ttagtgactg tcattttttt tcatctctcc acttctccta ataggtttgg cagtgtcaac catctcatca
ttctctccac ctgggtacca agtctctttc tgcttggtga tggtactcct ttttgcagtg gacacaggac
tatatttctc tgtgaagaca aacatttgaa gctcaacaag agactggaag gaccataaac ttaaatggag
aaaggaccct caagacaaat gacccccatc ccatgggggt aataagagca gtggcagcag catctctgaa
catttctctg gatttgcaac cccatcatcc tcaggcctct ctacaagcag caggaaacat agaactcaga
gccagatcct ttatccaact ctcgattttt ccttggtctc cagtggaagg gaaaagccca tgatcttcaa
gcagggaagc cccagtgagt agctgcattc ctagaaattg aagtttcaga gctacacaaa cactttttct
gtcccaacca ttccctcaca gcaaagcaac aatacaggct agggatggta atcctttaaa catacaaaaa
ttgctcgtat tataaattac ccagtttaga gggaaaaaaa gaaaataatt attcctaaac aaatggataa
gtagaattaa tggttgaggc aggaccctac agagtgtggg aactgctggg gatctagaga attcagtggg
accaatgaaa gcatggctga gaaatagcag ggtagtccag gatagtctaa gggaggtgtt cccatctgag
cccagagata agggtgtctt cctagaacat tagccgtagt ggaattaaca ggaaatcatg agggtgacgt
agaattgagt cttccagggg actctatcag aactggacca tttccaagta tataacgatg agtcctctaa
tgctaggagt agaaaatggt cctaggaagg ggactgagga ttggggtggg ggtggggtgg aaaagaaagt
acagaacaaa ccctgtgtca ctgtctcaag ttaagctaag tgaacagaac tatctcagca tcagaatgag
aaagcctgag aagaaagaac caaccagaag cacacaggaa ggaaagcgca ggaggtgaaa atgctttctt
ggccggggta gtaagaatta gaggttaatg cagggactgt aaaaccacct tttctgcttc aatgtctagt
tcctgtatag ctttgttcat tgcatttatt aaacaaatgt tgtataacca atactaaatg tactactgag
cttcactgag ttacgctgtg aaactttcaa atccttcttc atgtcagttc caatgaggtg gggatggaga
agacaattgt tgcttatgaa agaaagcttt agctgtctct gttttgtaag ctttcagtgc aacatttctt
ggttccaata aagcatttta caagatcttg catgctactc ttagatagaa gatggcaaaa ccatggtaat
aaaatatgaa tgataaaa (SEQ ID NO: 3)
MWQLLLPTALLLLVSAGMRTEDLPKAVVFLEPQWYSVLEKDSVTLKCQGAYSPEDNSTQWFHNENLISSQASSYFID
AATVNDSGEYRCQTNLSTLSDPVQLEVHIGWLLLQAPRWVFKEEDPIHLRCHSWKNTALHKVTYLQNGKDRKYFHHN
SDFHIPKATLKDSGSYFCRGLVGSKNVSSETVNITITQGLAVSTISSFSPPGYQVSFCLVMVLLFAVDTGLYFSVKT
NI (SEQ ID NO: 4)

What is claimed is:

1. A method of determining the risk of an individual for developing systemic lupus erythematosus (SLE) and/or rheumatoid arthritis (RA), comprising:
providing a biological sample from the individual, wherein the biological sample comprises DNA; and
determining the copy number of the FCGR3A gene and/or the FCGR3B gene in the individual,
wherein a copy number of the FCGR3A gene of less than two is statistically significantly associated with an increased risk of the individual developing SLE and/or RA, and wherein a copy number of the FCGR3B gene of less than two is statistically significantly associated with an increased risk of the individual developing SLE.

2. The method of claim 1, wherein the determining step uses PCR.

3. The method of claim 2, wherein the determining step uses real-time PCR.

4. The method of claim 2, wherein the primer sequences for determining the copy number of the FCGR3A gene are shown in SEQ ID NOs: 5 and 6.

5. The method of claim 2, wherein the primer sequences for determining the copy number of the FCGR3B gene are shown in SEQ ID NOs: 8 and 9.

6. The method of claim 3, wherein the probe sequence for determining the copy number of the FCGR3A gene is shown in SEQ ID NO:7.

7. The method of claim 3, wherein the probe sequence for determining the copy number of the FCGR3B gene is shown in SEQ ID NO:10.

8. The method of claim 1, wherein the individual is Asian or of Asian descent.

9. The method of claim 1, wherein the individual is Taiwanese or of Taiwanese decent.

10. The method of claim 1, further comprising administering an effective amount of a therapeutic compound to the individual.

11. A method of determining the copy number of FCGR3A and/or FCGR3B in an individual, comprising:
providing a biological sample from the individual, and
determining the copy number of FCGR3A and/or FCGR3B in the biological sample.

12. The method of claim 11, wherein a low copy number of FCGR3A indicates a statistically significantly increased risk factor for the individual developing an autoimmune disease.

13. The method of claim 12, wherein the autoimmune disease is SLE and/or RA.

14. The method of claim 11, wherein a low copy number of FCGR3B indicates a statistically significantly increased risk factor for the individual developing an autoimmune disease.

15. The method of claim 14, wherein the autoimmune disease is SLE.

16. The method of claim 11, further comprising administering an effective amount of a therapeutic compound to the individual.

17. A method of determining the risk of an individual for developing an autoimmune disease, comprising:
providing a biological sample from the individual; and
determining the copy number of the FCGR3A gene and/or the FCGR3B gene in the biological sample,
wherein a low copy number of the FCGR3A gene and/or the FCGR3B gene is indicative of a statistically significantly increased risk for the individual to develop an autoimmune disease.

18. The method of claim 17, wherein the autoimmune disease is SLE and/or RA.

19. The method of claim 17, wherein low copy number refers to less than two copies.

20. The method of claim 17, further comprising administering an effective amount of a therapeutic compound to the individual.


SEQUENCE LISTING (10 sequences)

SEQ ID NO: 1 - DNA, 8865 bp, Homo sapiens
ggagccccggctcctaggctgacagaccagcccagatccagtggcccggaggggcctgagctaaatccgcaggacctgggtaacacgaggaaggtaaagagttcctgtcctcgcccctccccacccccaccttttctgtgatcttttcagcctttcgctggtgacttgttcttccagggcccatttctctaccctacctgggtttcttctaacctggaaatctaatgatcaaatcacactaaaaagtcagtagctcctgtggattacatatcccaggagcatatagattttgaattttgaattttgaaagaaattctgcgtggagataatattgaggcagagacactgctagtggtctgaagatttgaaaggaccactttctgtgtgcaggcagggcctcagctggagatagatgggtctgggcgaggcaggagagtgacaagttctgaggtgaaatgaaggaagccctcagagaatgctcctcccaccttgaatctcatccccagggtctcactgtcccattcttggtgctgggtggatccaaatccaggagatggggcaagcatcctgggatggctgagggcacactctggcagattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcatgtggcagctgctcctcccaactgctctgctacttctaggtaagtcagggtctccctggttgagggagaagtttgagatgccttgggttcagcagagaccccttttcaggctacgaatgagactcccacgaagggatgggacccctcaccacatctatagctgtggattgagctcctaggacaagccaagatggggctagaaatgaggagaatgctggttccaattggggcatactcatgagtgaggccagtcacttcacccctctgggtcccagaatcactctgtggaaccaaagagcttcgactagatggtccctagggtctgtctctttcagtttgacattccagggttctcctctatgattttcaatttctaccctttcttgtggggatatgggttgaggctctttctgtagcttggttcagggaaattcaacctgtacccttaatttgtgagtttgcacagggagcaaggggtaagggagcagtgttgaaaatagggatttgtgttgacagtggcgcaagaggcatgaacagtggagaccagagagcaggtagcaaggtttccaccagaaacatcctgattcttgggaaaattgggctcctggggcagaggagggcaggggagttttaaactcactctatgttctaatcactctgatctctgcccctactcaatatttgatttatcttttttcttgcagtttcagctggcatgcggactggtgagtcagcttcatggtcttggattgacccagtggggcacatatggggacaaaggccataagatattgggaaatgcttgttgaatgggaaaatgctgatgtggggttagcagggatagttcctccaacacagcagaacttggccctgtgcttctctggccagctttccttaagatactgaacaggccaaaaatggggccaagatgctctaagactgagccaccaagcatgggtttgcaatgagctcattctggctttgaggctccctgggaatggcagtgtagagcctgctcctctccctgtcctcaccccacattatcttggctcctcagaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggtgagttgatgaaggggaagaggaaaatcaccaataaagggtgaaacaaagggtcctgaaatacttggtaagagccagagatgatattcttagagataaaagctaagatgagatgatgtgtggtcccactgaatggtatcagagttgtagtcctagctctaagtaggtcttgggcaaaatgtcaaagcctgtcagacagtagatataggactgctgcattgcacaattccaagaatccccatatggagtgcatacaatgtgaatgtgtcatgtgaaggttaggccatggcatagatgctcaataatagttatttatatatttattttcattttttttaattttattttttgagacagagtatcactctgtcacccaggctggagtgcaatgcggcaatctcagctcactgcaacttctgcccccttgggttgtagtgattctcctgcctcagcctcccgagtagctgagattacaggcacccgccaccacgcccagctaatttttgtatttttagtagagacagggtttcaccatgttggtcagtctggtctcaaactcctgacctcaggtgattcaccagccttggcttcccaaagtgctgggactacaggcgtgagccaccacacctggccaataatatttattgaataaattaatgaatttggtgttaggacctcaatctccttctcgctctcagacatgtaatgccctaagccacctcccaaagcaatcctagtggcctagcatcatatctttctgtctcctcatcaatgctatactcaaacctataattaagcataaatttggtaatgtgatagctcttccaatagaggcagatacatgttcagcctgcacattaatcatgacatgaaagttcttgtgtactattaacagaatatagacgtcagacacaggtaggagaaatattttgaaggcagaggtctttcctggtgtccctacaatcttaccacataggctggtccctgcagtgtcgccctgcaaacctaactctacttccacggctgttccattcatacaatgtttatgggtggaacaagctttgggggaagaagggcataaggaggtggatctgcaagagagctccatggaattgggcctctgaaactgatttttgtggctctttggcctctgacagtaccactcaactgacatggtcttcactctccagagctacaagaagatatgtccatttctagctaggtaagagatgtccacctacaaccaaataaaatgggggaattaccaagagaaagcaatagaaaaatcaagtctaagagttactagtttgccttgaacttggctctagaaactggctttagaagtctagccaatcaaggctatattaaactgtgaccatgagaattagcttcaccaggtaaacttctgagcatcctttaatcctttaggacccatttcacttatgtcctcctctgagaagcattttttacttctttttttgtttgtttgtttgtgtttgtttttgtttttgtttttgagacagagtctctctctgtcacccaagctggagtgcagtggcgcaatcttggctcactgcaacctccacctcccgggttcaagcaattctcctgcctcagcctcccaagtagctgggactacaggtgcatgccaccacgcccggctaattttttgtatttttagtagagacagggtttcgcagcgttagccaggatggtcttgatctcctgacttcatgatctgcccacctcggcctcccaaagtactaggattacagatgtgagccaccgcgcccagcctgcattttttacttctttcaggcagaatttctttattccaatctagtcagccccgcagtcctttattcttagcctgttgtagcacttgtcatattgtattgtgattatttctgaatatttatgtttctatgtctagactgtagattctttgaggctgagaactatatgtcccatcatctgggtatctccagtccacagtgtgtcatacatagtgagtgcttgatgaaatatcacttgaaggaatatacatatggacattcactgggtccatgacaggatagattcgaacaagaatgttcctccaaaggccaccagactatatactaaccatgactttatgctaataatgattcatctctctgctgaaaaagtaagtggatagataggcacatggcttcttttgataaatgatatctcttaataggtaatgaagattactttctgtttggcaaatctttgtggtagagaatcatgaccaacacacgtcctaccaattttgtttagcatcaggtagtagattttttaaattatagtaattcaagctgagaatgtagatttaaaaaataaaattattgtaaattttgttttgttcttattacaaaagtcatttggggtcaatttcaaaaatatataaaagtaaacaggagaaatttaaaatgtccttcagtcccactccttcagagaaaacccctgttaatatgtaagtgcatatccttcttttttctgtgcataatactttttaaaatatttgaagtattatgcttttttaacttaaaattgtctcatgaatattttcttatgccattataatacttacctataacatcattattttttaattattcaggccctttcccgaccatgacctcatgttctctctttgtgaagtctgattacttggtgacatgatcgtgagaataagctctggcgatataagaatttcctctcttgaaggccatgctcagtaaattacttggtgacatgatcgtgagaataagctctggcgatacaagaatttcctctcttgaaggccatgctcagtaataaagttggtctcaccgaggccctgtgacaccttagaaaccacgaattgccaggctgagcaataccagtcccgcccttcccctccctggtgtttacattgagttctccttcacaatttctgcagccactccgtggccaccgtcaccttattcctgactgccacaagagtctttcaatattcctttgattgcctattccttctgaaatctaccttttcctctaatagggcaattcatcattttcaaatgcaatttttactctgatctagaacttactgtgaatccttgtcacctgccacagcaaatctaagtctagcacttaaggatcctgcagatatgctcatcgttgcttctcacttacctcattgcttagtccctctgctctaaccctgtgtgttgatcacatgtgtgtgtgtccctcttccccattagacaaaggtcttggtatgacttcagttctcttgcagggccccatcagctcttccccaaagggagctatgcagggttgactcccaatctggctttcccttatgtctcaggatctgggtggtacgtggccccttcacaaagctctgcactgagagctgaggcctcccgggcctggggtgtctgtgtctttcaggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtgagacatgtgccaccctggaatgcccagggacgcctgtgtgtggaacctgcaatcacactgggaagttgagttgggaggagattcctgattcttacacgcacttcttcatatgtggttccctcctggtgatcaccaggaggtccccaaaagtccctgattgcagggtaggtttgcagctctgtttcagtccattcttttggggtagctaggaggtgtcattcactctgcagcatgatggcaggagcagaagccacatctcctccccaataaatacctctgtctttccttacgctaatcacacccacggtgtcatatgttcctatcgtgctggcctccttcttatccaagccttttagccacgatccaaactggcaggagcccctcatcccctcacagaaagagcccagaacctgggttctggccctgcagctaattaaccatctgaccagaggtgagccacttagtctctctgaaccccaatttcttcttccgtaacaaaaataagctgacatttattgggcacctttcagtgtgctagactctgtgctaaacaattctttacatgcacctggtttgactatcacagtagaccttcacaacatgagataggtaatattccattttacagatgaagtaaccgaggtgcaaaaataaataaataagtttccctaaggtcacatcaaagacttcaaagcctgtatatttaaccagtaagtaaaagatttgaacaagcactaatatcctatgatcccattaagtcatccacaaaacatctctaggttctgtagcaccagcctccagaatcagagctctagagtggtgtgcctggactttccagtttcacagaacttctatctgtaactagcccaagacataaattgtaaacaatttgcatgtagaaaggcagcaaaacaccttttgagattttgacactacaatgccataatttgtacaaaaataatttcatgacactttaaactgaaagtaaatactcccaagtggttagggaaagagagcaaataaagcaaatggggtaacatgtaaacaatgagtggatctgggtaaaggatatacgagattaaactattctggtcattttttttttaagtttggaaatatatcaaaatcaagagtttaaaaaattgaaatgcaaaatcaacaaatttgtcccagtttctagaccatagcattgtctgacaatttcttaactgtcacacaaaacccagcttacaacctaacttgttaacgctccctgtcacatctctgtcaaacaagcaggagcctttgctcagtgtttggtgagctgtcctctgctcagatagcactaagatcaggaaccaatgggaggaagcaatactttcccccagacttccccaccattcctaccacttgcctgttggctgttgtcaaagactttctactggtgacctcactgtttgttccaaatatctgccttagtgactgtcattttttttcatctctccacttctcctaataggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaa

SEQ ID NO: 2 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 3 - DNA, 8768 bp, Homo sapiens
aggctgacagaccagcccagatccagtggcccggaggggcctgagctaaatccgcaggacctgggtaacacgaggaaggtaaagagttcctgtcctcacccctccccacccccaccttttctgtgatcttttcagcctttcactggtgacttgttcttccagggcccatttctctaccctacctgggtttcttctaacctggaaatctaatgatcaaatcacactaaaaagtcagctcctgtggattacatatcccaggagcatatagattttgaattttgaattttgaaagaaattctgcgtggagataatattgaggcagagacactgctagtggtcaaagatttgaaaggacaactttctgtgtgcaggcagggcctcggctggagatagatgggtctggacgaggcaggagagtgagaagttctgaggtgaaatgcaggaagccctcagagaatgctcctcccaccttgaatctcatccccagggtcttgctgtcccattcttggtgctgggtggatctaaatccaggagatgggggcaagcatcctgggaaagctgagggcacactctggcagattctgtgtgtgtcctcagatgctcagccgcagacctttgggagagtaaagggggcacacccacccaccttgcctccaggctctttccttcctattcctgttctatggtggggctccattgcgagacttcagattgagaaatcagatgaagtttcaagaaaaggaaactggcaggtgacagagatgggtggagggactggggaaaggctgtttactccctcctgtctagtcggcttggtccctttagggctccggatatctttggtgacttgtccactccagtgtggcatcatgtggcagctgctcctcccaactgctctgctacttctaggtaagtcaggatctccctggttgagggagaagtttgagatgccttgggttcatcagacaccccttttcaggctacgaatgagactcccacaaagggatgggacccctcaccacatctatagctgtggattgagctaccaggacaagccaagatggggctagaaatgaggagaatgctggttccaattgggtcatagtcatgagtgaggccagtcacttcacccctctgggtcccagaatcactatgtggaaccaaagagcttcgactagatggtccctagggtctgtctctttcagtttgacattccagggttctcctctatggtttttaatttctaccctttcttgtggggatatgggttgaggctctttctgtagcttggttcagggaaattcaacctgtacccttaatttgtgagtttgcacagggagcaaggggtaagggagcagtgttgaaaatagggatttgtgttgacagtggcgcaagaggcatgaacagtggagaccagagagcaggtagcaaggtttccaccagaaacatcctgattcttgggaaaattgggctcctggggcagaggagggcaggggagttttaaactcactctatgttctaatcactctgatctctgcccctactcaatatttgatttactcttttttcttgcagtttcagctggcatgcggactggtgagtcagcttcatggtcttggattgacccagtggggcacatatggggacaatggccataagatattgggaaatgcttgttgaatgggaaaatgctgatgtggggttagcagggatagttcctccaacacagcagaacttggccctgtgcttctctggccagctttccttaagatactgaacaggccaaaaatggggccaagatgctctaagactgagccaccaagcatgggtttgcaatgagctcattctggctttgaggctccctgggaatggcagtgtagagcctgctcctctccctgtcctcaccccacattatcttggctcctcagaagatctcccaaaggctgtggtgttcctggagcctcaatggtacagcgtgcttgagaaggacagtgtgactctgaagtgccagggagcctactcccctgaggacaattccacacagtggtttcacaatgagaacctcatctcaagccaggcctcgagctacttcattgacgctgccacagtcaacgacagtggagagtacaggtgccagacaaacctctccaccctcagtgacccggtgcagctagaagtccatatcggtgagttgatgaaggggaagaggaaaatcaccaataaagggtgaaacaaagggtcctgaaatacttggtaagagccagagatgatattcttagagataaaagctaagatgagatgatgtgtggtcccactgaatggtatcagagttgtagtcctagctctaagtaggtcttgggcaaaatgtcaaagcctgtcagacagtagatataggctgctgcattgcacaattccaagaatccccatatggagtgcatacaatgtgaatgtgtcatgtgaaggttaggccatggcatagatgctcaataatagttatttatttatttattttcattttttttaattttattttttgagacagagtatcactctctcacccaggctggagtgcaatgcggcaatctgagctcactgcaacttctgcccccttgggttgtagtgattctccagcctcagcctcccgagtagctgagattacaggcacccgccaccacgcccagctaatttttgtatttttagtagagacagggtttcaccatgttggtcagtctggtctcaaactcctgacctcaggtgattcaccggtcttggctttccaaagtgctgggactacaggcgtgagccaccacacctggccaataatttttattgaataaattaatgaatttggtgttaggacctcaatctccttctcgctctcagacatgtaatgccctaagccacctcccaaagcaatcctagtggcctagaatcatatctttctgtctcctcatcaatgctatactcaaacctataattaagcataaatttggtaatgtgatagctcttccaatagaggcagatacatgttcagcctgcacattaatcatgacatgaaagtccttgtgtactattaacagaatatagacatcagacacaggtaggagaaatattttgaaggcaggggtctttcctggtgtccctacaatcttaccacatagcctggtccctgcagtgtcgccctgcaaacctaattctacttccacggctgttccattcatacaatgtttatgggtggaacaagctttgggggaagaagggcataaggaggtggatctgcaagagagctccatggaattgggcctctgaaactgatttttgtggctctttggcctctgacattaccactcaactgacatggtcttcactctccagagctacaagaaggtatgtccatttctagctaggtaagagatgtccacctacaaccaaataaaatgggggaattaccaagagaaagcaatagaaaaatcaagtctaggagttactagtttgccttgaacttggctctagaaactggctttagaagtctagccaatcaaggctatattaaactgtgaccatgagaattagcttcaccaggtaaacttctgagcatcctttattcctttaggacccatttcacttatgtcctcctctgagaagcactttttacttctttgtttgtttgtttgtttttgtttttgtttttgagacagagtttctctctgtcacccaagctggagtgcagtggcgcaatcttggttcactgcaacctccacctccctggttccagcgattctcctgcttcagcctcccaactagctgggactacaggtgcatgccaccacgcccggctaattttttgtatttttagtagagacagggtttcacggcattagccaggatggtcttgatctcctgacctcgtgatctgcctacttcagcctcccaaagtactaggattacagatgtgagccaccgcgcccagcctgcattttttacttctttcaggcagaatttctttattccaatctagtcggccccgcagtcctttattcttagcctgttgtagcactcgtcatattgtattgtgattatttctgaatatttatgtttctatgtctaggctgtagattctttgaggctgagaactatatgtcccatcatctgggtatctccagtccacagtgtgtcatacatagtgagtgcttgatgaaatatcacttgaaggaatatacacatgggcattcactgagtccatgacaggacagattcgaacaagaatgttcctccaaaggccaccagactatatactaaccatgactttatgctaataatgattcatctctttgctgaaaaagtaagtggatagataggcacatggcttcttttgataaatgatatctcttaataggtaatgaagattactttctgtttggcaaatctttgtgttagagaatcatgaccaacacacgtcctaccaattttgtttagcatcaggtagtagatttttaaaattatagtaattcaacctgagaatgtagatttaaaaaataaaattacagtaaatttagttttgctcttattacgtttcgggtcaatttcaaaaatatataaaagtaaacaggagaaatttaaaatgtcctttggtcccactccttcagagaaaacccctgttaatatgtaagtgcatatccttcttttttctgtgcataatactttttaaaatatttgaagtattatgcttttttaacttaacattgtctcatgaatattttcttatgccattataatacttacctataacatcattatttttttaattattcaggccatttcccgaccatgacctcatgttctctctttgtgaagtctgattacttggtgacatgattgtgagaataagctctggcgatataagaatttcctctcttgaaggccatgctcagtaataaagttggtctcaccgaggccctgtgacaccttagaaaccacgaattgccactctgagcactaccagtcccgcccttcgccaccctgatgtttacattgagttagttctccttcacaatttctgcagccactctgtggcgaccgtcagcttattcctgaaggccacaagagtctttcaatattcctttgattgcctattccttctgaaatctaccttttcctctaatagagcaactcatcattttcaaatgcaatttttactctgatctagaacttactgtgaatccttgtcacctgccacagcaaatctaagtctagcacttaaggatcctgcagatatgctcatcgttgcttctcacttacctcattgcttagtccctctgctctaaccctgtgtgttgatcacatgtgtgtgtgtccctcttccccattagacaaaggtcttggtatgacttcagttctcttgcagggccccatcagctcttccccaaagggagctatgcagggttgactcccaatctggctttcccttatgcctcaggatctgggtggtacgtggccccttcacaaagctctgcactgagagctgaggcctcccgggcctggggtgtctctgtgtctttcaggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtgagacatgtgccaccctggaatgcccagggacgcctgtgtgtggaacctgcactcacactgggaagttgagttgggaggagattcctgattcttacacgcacttcttcatatgtggttccctcctggtgatcaccaggaggtccccaaaagtccctgattgcagggtaggtttgcagctctgtttcagtccattcttttggggtagctaggaggtgtcattcactctgcagcatgatggcaggagcagaagccacatctcctccccaataaatacctctgtctttccttaccctaatcacacccacggtgtcatatgttcctatcgtgctggcctccttcttatccaagccttttagccacgatccaaactggcaggagcccctcatcccctcacagaaagagcccagaacctgggttctggccctgcagctaattaaccatctgaccagaggtgagccacttagtctctctgaaccccaatttcttcttccgtaacaaaaataagctgacatttattgggcacctttcagtgtgctagactctgtgctaaacaattctttacatgcacctggtttgactaccacagtagaccttcacaacatgagataggtaatattccattttacagatgaagtaaccgaggtgcaaaaataaataaataagttgcccctaaggtcacatcaaagacttcaaagcctgtatatttaaccagtaagtaaaagatttgaagaagcactaatatcctatgatcccattaagtcatccacaaaacatctctaggttccatagcaccagcctccagaatcagagctctagagtggtgtgcctggactttccagtttcacagaacttctatctgtaactagcccaagacataaattgtaaacaatttgcatgtagaaaggcagcaaaacaccttttgagattttgacaccacaatgccataatttgtacaaaaataatttcatgacactttaaactgaaagtaaatactcccaagtggttagggaaagagagcaaataaagcaaatggggtaacatgtaaacaatgagtggatctgggtaaaggatatacgagatttctttatactattctggtcattttttttaagtttggaaatatatcaaaatcaagagtttaaaaaattgaaatgcaaaatcaacaaatttatcccagtttctagaccatagcattgtctgacaatttcttaactgtcacacaaaacccagcttacaacctaacttgttaacgctccctgtcacatctctgtcaaacaagcaggagcctttgctaagtgtttggtgagctgtcctctgctcagatagcactaagatcaggaaccaatgggaggaagcaatactttcccccagacctccccaccattcctaccacttgcctgttggcacttgtcaaagactctctactggtgacctcactgtttgttccaaatatctgccttagtgactgtcattttttttcatctctccacttctcctaataggtttggcagtgtcaaccatctcatcattctctccacctgggtaccaagtctctttctgcttggtgatggtactcctttttgcagtggacacaggactatatttctctgtgaagacaaacatttgaagctcaacaagagactggaaggaccataaacttaaatggagaaaggaccctcaagacaaatgacccccatcccatgggggtaataagagcagtggcagcagcatctctgaacatttctctggatttgcaaccccatcatcctcaggcctctctacaagcagcaggaaacatagaactcagagccagatcctttatccaactctcgatttttccttggtctccagtggaagggaaaagcccatgatcttcaagcagggaagccccagtgagtagctgcattcctagaaattgaagtttcagagctacacaaacactttttctgtcccaaccattccctcacagcaaagcaacaatacaggctagggatggtaatcctttaaacatacaaaaattgctcgtattataaattacccagtttagagggaaaaaaagaaaataattattcctaaacaaatggataagtagaattaatggttgaggcaggaccctacagagtgtgggaactgctggggatctagagaattcagtgggaccaatgaaagcatggctgagaaatagcagggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatttccaagtatataacgatgagtcctctaatgctaggagtagaaaatggtcctaggaaggggactgaggattggggtgggggtggggtggaaaagaaagtacagaacaaaccctgtgtcactgtctcaagttaagctaagtgaacagaactatctcagcatcagaatgagaaagcctgagaagaaagaaccaaccagaagcacacaggaaggaaagcgcaggaggtgaaaatgctttcttggccggggtagtaagaattagaggttaatgcagggactgtaaaaccaccttttctgcttcaatgtctagttcctgtatagctttgttcattgcatttattaaacaaatgttgtataaccaatactaaatgtactactgagcttcactgagttacgctgtgaaactttcaaatccttcttcatgtcagttccaatgaggtggggatggagaagacaattgttgcttatgaaagaaagctttagctgtctctgttttgtaagctttcagtgcaacatttcttggttccaataaagcattttacaagatcttgcatgctactcttagatagaagatggcaaaaccatggtaataaaatatgaatgataaaa

SEQ ID NO: 4 - protein, 233 aa, Homo sapiens
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 5 - DNA, 21 bp, Artificial Sequence
  synthetic oligonucleotide
ttcaagaaaaggaaattggtg

SEQ ID NO: 6 - DNA, 18 bp, Artificial Sequence
  synthetic oligonucleotide
gaggagcagctgccacat

SEQ ID NO: 7 - DNA, 16 bp, Artificial Sequence
  synthetic oligonucleotide
tccactccagtgtggc

SEQ ID NO: 8 - DNA, 21 bp, Artificial Sequence
  synthetic oligonucleotide
ttcaagaaaaggaaactggca

SEQ ID NO: 9 - DNA, 19 bp, Artificial Sequence
  synthetic oligonucleotide
gaggagcagctgccacatg

SEQ ID NO: 10 - DNA, 16 bp, Artificial Sequence
  synthetic oligonucleotide
tccactccagtgtggc